(19) United States
(12) Patent Application Publication
(10) Pub. No.: US 2006/0094719 A1
(43) Pub. Date: May 4, 2006

(54) Tetralin histamine-3 receptor antagonists

(75) Inventors: Harry R. Howard JR., Bristol, CT (US); Mark L. Elliott, Canterbury, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/256,339

(22) Filed: Oct. 21, 2005

Related U.S. Application Data
(60) Provisional application No. 60/623,702, filed on Oct. 29, 2004.

(51) Int. Cl. A61K 31/5377; A61K 31/496; A61K 31/454; C07D 413/02; C07D 403/02; C07D 401/02

(52) U.S. Cl. 514/235.2; 514/319; 514/326; 544/128; 546/206; 514/253.01; 544/360

(57) ABSTRACT

This invention is directed to compounds of the formula I 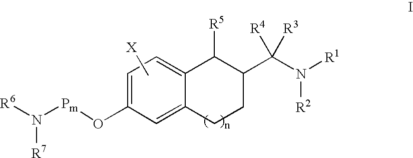 as defined herein, or a pharmaceutically acceptable salt thereof; a pharmaceutical composition containing a compound of formula I, a method of treatment of a disorder or condition that may be treated by antagonizing histamine H3 receptors, the method comprising administering to a mammal in need of such treatment a compound of formula I as described above, and a method of treatment of a disorder or condition selected from the group consisting of depression, mood disorders, schizophrenia, anxiety disorders, Alzheimer's disease, attention-deficit disorder (ADD), attention-deficit hyperactivity disorder (ADHD), psychotic disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastro-intestinal tract, the method comprising administering to a mammal in need of such treatment a compound of formula I as described above.

### BACKGROUND OF THE INVENTION

This invention is directed to compounds of formula I described herein, to a pharmaceutical composition comprising such compounds, and to methods of treatment of disorders or conditions that may be treated by antagonizing histamine-3 (H3) receptors using such compounds. The histamine-3 (H3) receptor antagonists of the invention are useful for treating anxiety disorders, including, for example, generalized anxiety disorder, panic disorder, PTSD, and social anxiety disorder; mood adjustment disorders, including depressed mood, mixed anxiety and depressed mood, disturbance of conduct, and mixed disturbance of conduct and depressed mood; age-associated learning and mental disorders, including Alzheimer's disease; attention adjustment disorders, such as attention-deficit disorders, or other cognitive disorders due to general medical conditions; attention-deficit hyperactivity disorder; psychotic disorders including schizoaffective disorders and schizophrenia; sleep disorders, including narcolepsy and enuresis; obesity; dizziness, epilepsy, and motion sickness. The H3 receptor antagonists of the invention are also useful for treating, for example, allergy, allergy-induced airway (e.g., upper airway) responses, congestion (e.g., nasal congestion), hypotension, cardiovascular disease, diseases of the GI tract, hyper- and hypo-motility and acidic secretion of the gastrointestinal tract, sleeping disorders (e.g., hypersomnia, somnolence, and narcolepsy), attention deficit hyperactivity disorder ADHD), hypo- and hyper-activity of the central nervous system (for example, agitation and depression), and other CNS disorders (such as schizophrenia and migraine).

Histamine is a well-known mediator in hypersensitive reactions (e.g. allergies, hay fever, and asthma) that are commonly treated with antagonists of histamine or “antihistamines.” It has also been established that histamine receptors exist in at least two distinct types, referred to as H1 and H2 receptors.

A third histamine receptor (H3 receptor) is believed to play a role in neurotransmission in the central nervous system, where the H3 receptor is thought to be disposed presynaptically on histaminergic nerve endings (Nature, 302, S32-837 (1983)). The existence of the H3 receptor has been confirmed by the development of selective H3 receptor agonists and antagonists (Nature, 327, 117-123 (1987)) and has subsequently been shown to regulate the release of the neurotransmitters in both the central nervous system and peripheral organs, particularly the lungs, cardiovascular system and gastrointestinal tract.

A number of diseases or conditions may be treated with histamine-3 receptor ligands wherein the H3 ligand may be an antagonist, agonist or partial agonist, see: (Imamura et al., Circulation Res., (1996) 78, 475-481); (Imamura et al., Circ. Res., (1996) 78, 863-869); (Lin et al., Brain Res. (1990) 523, 325-330); (Monti et al., Neuropsychopharmacology (1996) 15, 31-35); (Sakai et al., Life Sci. (1991) 48, 2397-2404); (Mazurkiewiez-Kwilecki and Nsonwah, Can. J. Physiol. Pharmacol. (1989) 67, 75-78); (Panula, P. et al., Neuroscience (1998) 44, 465-481); (Wada et al., Trends in Neuroscience (1991) 14,415); (Monti et al., Eur. J. Pharmacol. (1991) 205, 283); (Haas et al., Behav. Brain Res. (1995) 66, 41-44); (De Almeida and Izquierdo, Arch. Int. Pharmacodyn. (1986) 283, 193-198); (Kamei et al., Psychopharmacology (1990) 102, 312-318); (Kamei and Sakata, Japan. J. Pharmacol. (199 1) 57, 437-482); (Schwartz et al., Psychopharmacology; The Fourth Generation of Progress, Bloom and Kupfer (eds.), Raven Press, New York, (1995) 3, 97); (Shaywitz et al., Psychopharmacology (1984) 82, 73-77); (Dumery and Blozovski, Exp. Brain Res. (1987) 67, 61-69); (Tedford et al., J. Pharmacol. Exp. Ther. (1995) 275, 598-604); (Tedford et al., Soc. Neurosci. Abstr. (1996) 22, 22); (Yokoyama et al., Eur. J. Pharmacol. (1993) 234, 129); (Yokoyama and Iinuma, CNS Drugs (1996) 5, 321); (Onodera et al., Prog. Neurobiol. (1994) 42, 685); (Leurs and Timmerman, Prog. Drug Res. (1992) 39, 127); (The Histamine H3 Receptor, Leurs and Timmerman (ed.), Elsevier Science, Amsterdam, The Netherlands (1998); (Leurs et al., Trends in Pharm. Sci. (1998) 19, 177-183); (Phillips et al., Annual Reports in Medicinal Chemistry (1998) 33, 31-40); (Matsubara et al., Eur. J. Pharmacol. (1992) 224, 145); (Rouleau et al., J. Pharmacol. Exp. Ther. (1997) 281, 1085); (Adam Szelag, “Role of histamine H3-receptors in the proliferation of neoplastic cells in vitro”, Med. Sci. Monit., 4(5): 747-755, (1998)); (Fitzsimons, C., H. Duran, F. Labombarda, B. Molinari and E. Rivera, “Histamine receptors signalling in epidermal tumor cell lines with H-ras gene alterations”, Inflammation Res., 47 (Suppl. 1): S50-S51, (1998)); (R. Leurs, R. C. Vollinga and H. Timmerman, “The medicinal chemistry and therapeutic potentials of ligand of the histamine H3 receptor”, Progress in Drug Research 45: 170-165, (1995)); (R. Levi and N. C. E. Smith, “Histamine H3-receptors: A new frontier in myocardial ischemia”, J. Pharm. Exp. Ther., 292: 825-830, (2000)); (Hatta, E., K Yasuda and R. Levi, “Activation of histamine H3 receptors inhibits carrier-mediated norepinephrine release in a human model of protracted myocardial ischemia”, J. Pharm. Exp. Ther., 283: 494-500, (1997); (H. Yokoyama and K. Iinuma, “Histamine and Seizures: Implications for the treatment of epilepsy”, CNS Drugs, 5(5); 321-330, (1995)); (K. Hurukami, H. Yokoyama, K. Onodera, K. Iinuma and T. Watanabe, AQ-0 145, “A newly developed histamine H3 antagonist, decreased seizure susceptibility of electrically induced convulsions in mice”, Meth. Find. Exp. Clin. Pharmacol., 17(C): 70-73, (1995); (Delaunois A., Gustin P., Garbarg M., and Ansay M., “Modulation of acetylcholine, capsaicin and substance P effects by histamine H3 receptors in isolated perfused rabbit lungs”, European Journal of Pharmacology 277(2-3):243-50, (1995)); and (Dimitriadou, et al., “Functional relationship between mast cells and C-sensitive nerve fibres evidenced by histamine H3-receptor modulation in rat lung and spleen”, Clinical Science 87(2):151-63, (1994). Such diseases or conditions include cardiovascular disorders such as acute myocardial infarction; memory processes, dementia and cognition disorders such as Alzheimer's disease and attention deficit hyperactivity disorder; neurological disorders such as Parkinson's disease, schizophrenia, depression, epilepsy, and seizures or convulsions; cancer such as cutaneous carcinoma,” medullary thyroid carcinoma and melanoma; respiratory disorders such as asthma; sleep disorders such as narcolepsy; vestibular dysfunction such as Meniere's disease; gastrointestinal disorders, inflammation, migraine, motion sickness, obesity, pain, and septic shock.

H3 receptor antagonists have also been previously described in, for example, WO 03/050099, WO 02/0769252, and WO 02/12224. The histamine H3 receptor (H3R) regulates the release of histamine and other neurotransmitters, including serotonin and acetylcholine. H3R is relatively neuron specific and inhibits the release of certain monoamines such as histamine. Selective antagonism of H3R raises brain histamine levels and inhibits such activities as food consumption while minimizing non-specific peripheral consequences. Antagonists of the receptor increase synthesis and release of cerebral histamine and other monoamines. By this mechanism, they induce a prolonged wakefulness, improved cognitive function, reduction in food intake and normalization of vestibular reflexes. Accordingly, the receptor is an important target for new therapeutics in Alzheimer's disease, mood and attention adjustments, including attention deficit hyperactive disorder (ADHD), cognitive deficiencies, obesity, dizziness, schizophrenia, epilepsy, sleeping disorders, narcolepsy and motion sickness, and various forms of anxiety.

The majority of histamine H3 receptor antagonists to date resemble histamine in possessing an imidazole ring that may be substituted, as described, for example, in WO 96/38142. Non-imidazole neuroactive compounds such as beta histamines (Arrang, Eur. J. Pharm. 1985, 111:72-84) demonstrated some histamine H3 receptor activity but with poor potency. EP 978512 and EP 982300 disclose non-imidazole alkyamines as histamine H3 receptor antagonists. WO 02/12224 (Ortho McNeil Pharmaceuticals) describes non-imidazole bicyclic derivatives as histamine H3 receptor ligands, and EP 1275647 (Les Laboratoires Servier) has disclosed novel octahydro-2H-pyrido[1,2-a]pyrazines that are selective H3 receptor antagonists. Other receptor antagonists have been described in WO 02/32893 and WO 02/06233.

This invention is directed to histamine-3 (H3) receptor antagonists of the invention useful for treating the conditions listed in the preceding paragraphs. The compounds of this invention are highly selective for the H3 receptor (vs. other histamine receptors), and possess remarkable drug disposition properties (pharmacokinetics). In particular, the compounds of this invention selectively distinguish H3R from the other receptor subtypes H1R, H2R. In view of the increased level of interest in histamine H3 receptor agonists, inverse agonists and antagonists in the art, novel compounds that interact with the histamine H3 receptor would be a highly desirable contribution to the art. The present invention provides such a contribution to the art being based on the finding that a novel class of tetralin compounds exhibits a high and specific affinity to the histamine H3 receptor.

### SUMMARY OF THE INVENTION

This invention is directed to compounds of the formula I: 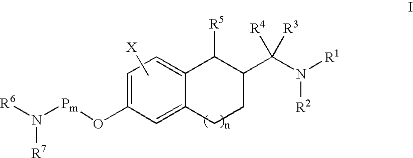
or the pharmaceutically acceptable salt(s) thereof, wherein:

m=2, 3 or 4;

n=0, 1 or 2;

P is independently selected from the group consisting of CH₂, C₂-C₆ alkyl or cycloalkyl group optionally substituted by hydrogen, C₁-C₆ alkyl or OH;

R¹ and R² together with the nitrogen to which they are attached form a 3-10 member cyclic or bicyclic ring, optionally substituted with up to two additional heteroatoms selected from the group consisting of N, O or S [including the cyclic or bicyclic rings azetidine, pyrrolidine, piperidine, azepine piperazine, morpholine and thiomorpholine] and optionally substituted with H, C₁-C₆ alkyl or cycloalkyl groups, aryl or heteroaryl rings, or oxygen (including sulfoxide or sulfone); R¹ and R² are independently selected from the group that includes hydrogen, C₁-C₆ alkyl or C₃-C₇ cycloalkyl;

R³, R⁴ and R⁵ are independently selected from the group consisting of hydrogen, and C₁-C₆ alkyl;

R⁶ and R⁷ are independently selected from the group consisting of hydrogen, C₁-C₁₂ alkyl, C₃-C₈ cycloalkyl, aryl or heteroaryl optionally substituted with up to three of Y; or

R⁶ and R⁷ together with the nitrogen to which they are attached form a 3-10 member cyclic or bicyclic ring optionally substituted with up to two additional heteroatoms selected from N, O or S [including the cyclic or bicyclic rings azetidine, pyrrolidine, piperidine, azepine, piperazine, morpholine and thiomorpholine] and optionally substituted by up to three groups of R²¹, O(═C)—R²¹ and CONR²²R²³

wherein R²¹, R²² and R²³ are independently selected from the group consisting of H, C₁-C₆ alkyl, aryl, C₃-C₈ cycloalkyl, hydroxyl, hydroxyalkyl, alkoxyalkyl, arylalkyl, heteroarylalkyl; and

X and Y are independently selected from the group consisting of H, F, Cl, Br, I, CN, OH, NR¹⁵R¹⁶, CF₃, C₂F₅, C₁-C₆ alkyl or C₃-C₈ cycloalkyl, aryl and heteroaryl [optionally substituted with halogen, C₁-C₆ alkyl, C₁-C₆ alkoxy, CF₃], C₁-C₆ alkoxy, and C₁-C₆alkyl-S(O)_{q}—, wherein q is 0, 1, 2 and R¹⁵ and R¹⁶ are independently selected from R¹ and R².

Where cis and trans isomers are possible for an embodiment of the inventive compound of formula I, both cis and trans isomers are within the scope of the invention.

The term “alkyl” refers to straight or branched chains of carbon atoms. Exemplary alkyl groups are C₁-C₆ alkyl groups which include methyl, ethyl, propyl, isopropyl, butyl, isobutyl, pentyl, isopentyl, hexyl, and the like, including all regioisomeric forms thereof, and straight and branched chain forms thereof. The term “alkyl” is also used to denote straight or branched chains of carbon atoms having one or more carbon-carbon double bonds, such as vinyl, allyl, butenyl, and the like, as well as straight or branched chains of carbon atoms having one or more carbon-carbon triple bonds, such as ethynyl, propargyl, butynyl, and the like. The term “aryl” denotes a cyclic, aromatic hydrocarbon. Examples of aryl groups include phenyl, naphthyl, anthracenyl, phenanthrenyl, and the like. The terms “alkoxy” and “aryloxy” denote “O-alkyl” and “O-aryl”, respectively. The term “cycloalkyl” denotes a cyclic group of carbon atoms, where the ring formed by the carbon atoms may be saturated or may comprise one or more carbon-carbon double bonds in the ring. Examples of cycloalkyl groups include cyclopropyl, cyclobutyl, cyclopentyl, cyclohexyl, cycloheptyl, and the like, as well as cyclopentenyl, cyclopentadienyl, cyclohexenyl, cyclohexadienyl, and the like. As used herein, the term “cycloalkyl” is also intended to denote a cyclic group comprising at least two fused rings, such as adamantanyl, decahydronaphthalinyl, norbornanyl, where the cyclic group may also have one or more carbon-carbon double bonds in one or both rings, such as in bicyclo[4.3.0]nona-3,6(1)-dienyl, dicyclopentadienyl, 1,2,3,4=tetrahydronaphthalinyl (tetralinyl), indenyl, and the like. The term “halogen” represents chloro, fluoro, bromo, and iodo. The term “heteroaryl” denotes a monocyclic or bicyclic aromatic group wherein one or more carbon atoms are replaced with heteroatoms selected from the group consisting of nitrogen, oxygen, and sulfur. If the heteroaryl group contains more than one heteroatom, the heteroatoms may be the same or different. Preferred heteroaryl groups are five- to fourteen-member rings that contain from one to three heteroatoms independently selected from oxygen, nitrogen, and sulfur. Examples of preferred heteroaryl groups include benzo[b]thienyl, chromenyl, furyl, imidazolyl, indazolyl, indolizinyl, indolyl, isobenzofuranyl, isoindolyl, isoquinolyl, isothiazolyl, isoxazolyl, naphthyridinyl, oxadiazolyl, oxazinyl, oxazolyl, phthalazinyl, pteridinyl, purinyl, pyranyl, pyrazinyl, pyrazolyl, pyridazinyl, pyridyl, pyrimidyl, pyrrolyl, quinolizinyl, quinolyl, quinoxalinyl, thiazolyl, thienyl, triazinyl, triazolyl, and xanthenyl.

The term “heterocycloalkyl” denotes a cycloalkyl system, wherein “cycloalkyl” is defined above, in which one or more of the ring carbon atoms are replaced with a heteroatom selected from the group consisting of nitrogen, oxygen, and sulfur. Examples of such heterocycloalkyl groups include azabicycloheptanyl, azetidinyl, benzazepinyl, 1,3-dihydroisoindolyl, indolinyl, tetrahydrofuryl, tetrahydroquinolinyl, tetrahydroisoquinolinyl, morpholinyl, piperazinyl, piperidyl, pyrrolidinyl, and, tetrahydro-2H-1,4-thiazinyl.

A cyclic group may be bonded to another group in more than one way. If no particular bonding arrangement is specified, then all possible arrangements are intended. For example, the term “pyridyl” includes 2-, 3-, or 4-pyridyl, and the term “thienyl” includes 2- or 3-thienyl.

The term “C₀-C₄” includes the embodiment where there are no carbons in a chain. Thus, for example, the groups “C₃-C₇ cycloalkyl-C₀-C₄ alkyl,” “C₆-C₁₄ aryl-C₀-C₄ alkyl,” “5-10-membered heteroaryl-C₀-C₄ alkyl,” and “C₆-C₁₄ aryl-C₀-C₄ alkylene-O-C₀-C₄ alkyl” include C₃-C₇ cycloalkyl, C₆-C₁₄ aryl, 5-10-membered heteroaryl, and C₆-C₁₄ aryl-O-C₀-C₄ alkyl, respectively.

The term “C₁-C₄ dialkylamino” refers to a dialkylamino group in which each alkyl group is independently a C₁-C₄ alkyl group.

This invention is also directed to:

a pharmaceutical composition for treating, for example, a disorder or condition that may be treated by antagonizing histamine-3 receptors, the composition comprising a compound of formula I as described above, and optionally a pharmaceutically acceptable carrier;

a method of treatment of a disorder or condition that may be treated by antagonizing histamine-3 receptors, the method comprising administering to a mammal in need of such treatment a compound of formula I as described above; and

a pharmaceutical composition for treating, for example, a disorder or condition selected from the group consisting of depression, mood disorders, schizophrenia, anxiety disorders, Alzheimer's disease, attention deficit disorder (ADD), attention deficit hyperactivity disorder (ADHD), psychotic disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastrointestinal tract, the composition comprising a compound of formula I as described above, and optionally a pharmaceutically acceptable carrier.

This invention is also directed to a method of treatment of a disorder or condition selected from the group consisting of the disorders or conditions listed in the preceding paragraph, the method comprising administering to a mammal in need of such treatment a compound of formula I as described above.

The histamine-3 (H3) receptor antagonists of the invention are useful for treating, in particular, ADD, ADHD, obesity, anxiety disorders and respiratory diseases. Respiratory diseases that may be treated by the present invention include adult respiratory distress syndrome, acute respiratory distress syndrome, bronchitis, chronic bronchitis, chronic obstructive pulmonary disease, cystic fibrosis, asthma, emphysema, rhinitis and chronic sinusitis.

The pharmaceutical composition and method of this invention may also be used for preventing a relapse in a disorder or condition described in the previous paragraphs. Preventing such relapse is accomplished by administering to a mammal in need of such prevention a compound of formula I as described above.

The disclosed compounds may also be used as part of a combination therapy, including their administration as separate entities or combined in a single delivery system, which employs an effective dose of a histamine H3 antagonist compound of general formula I and an effective dose of a histamine H1 antagonist, such as cetirizine (Zyrtec™), for the treatment of allergic rhinitis, nasal congestion and allergic congestion.

The disclosed compounds may also be used as part of a combination therapy, including their administration as separate entities or combined in a single delivery system, which employs an effective dose of a histamine H3 antagonist compound of general formula I and an effective dose of a neurotransmitter reuptake blocker. Examples of neurotransmitter reuptake blockers will include the serotonin-selective reuptake inhibitors (SSRI's) like sertraline (Zoloft™), fluoxetine (Prozac™), and paroxetine (Paxil™), or non-selective serotonin, dopamine or norepinephrine reuptake inhibitors for treating depression and mood disorders.

The compounds of the present invention may have optical centers and therefore may occur in different enantiomeric configurations. Formula I, as depicted above, includes all enantiomers, diastereomers, and other stereoisomers of the compounds depicted in structural formula I, as well as racemic and other mixtures thereof. Individual isomers can be obtained by known methods, such as optical resolution, optically selective reaction, or chromatographic separation in the preparation of the final product or its intermediate.

The present invention also includes isotopically labeled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula I of this invention and prodrugs thereof can generally be prepared by carrying out the procedures disclosed in the Schemes and/or in the Examples and Preparations below, by substituting a readily available isotopically labeled reagent for a non-isotopically labeled reagent.

“Antagonizing histamine-3 (H3) receptors,” as used herein, refers to acting as a histamine-3 receptor antagonist.

A “unit dosage form” as used herein is any form that contains a unit dose of the compound of formula I. A unit dosage form may be, for example, in the form of a tablet or a capsule. The unit dosage form may also be in liquid form, such as a solution or suspension.

The compositions of the present invention may be formulated in a conventional manner using one or more pharmaceutically acceptable carriers. Thus, the active compounds of the invention may be formulated for oral, buccal, intranasal, parenteral (e.g., intravenous, intramuscular or subcutaneous) or rectal administration or in a form suitable for administration by inhalation or insufflation.

For oral administration, the pharmaceutical compositions may take the form of, for example, tablets or capsules prepared by conventional means with pharmaceutically acceptable excipients such as binding agents (e.g., pre-gelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

The active compounds of the invention may be formulated for parenteral administration by injection, including using conventional catheterization techniques or infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insulator may be formulated containing a powder mix of a compound of the invention and a suitable powder base such as lactose or starch.

A proposed dose of the active compounds of the invention for oral, parenteral or buccal administration to the average adult human for the treatment of the conditions referred to above (e.g., depression) is 0.1 to 200 mg of the active ingredient per unit dose which could be administered, for example, 1 to 4 times per day.

Aerosol formulations for treatment of the conditions referred to above (e.g., attention deficit hyperactivity disorder) in the average human are preferably arranged so that each metered dose or “puff” of aerosol contains 20 μg to 1000 μg of the compound of the invention. The overall daily dose with an aerosol will be within the range 100 μg to 100 mg. Administration may be several times daily, for example 2, 3, 4 or 8 times, giving for example, 1, 2 or 3 doses each time.

In connection with the use of an active compound of this invention with a histamine H1 antagonist, preferably cetirizine, for the treatment of subjects possessing any of the above conditions, it is to be noted that these compounds may be administered either alone or in combination with pharmaceutically acceptable carriers by either of the routes previously indicated, and that such administration can be carried out in both single and multiple dosages. More particularly, the active combination can be administered in a wide variety of different dosage forms, i.e., they may be combined with various pharmaceutically acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of formula I are present in such dosage forms at concentration levels ranging from about 0.5% to about 95% by weight of the total composition, i.e., in amounts which are sufficient to provide the desired unit dosage and a histamine H1 antagonist, preferably cetirizine, is present in such dosage forms at concentration levels ranging from about 0.5% to about 95% by weight of the total composition, i.e., in amounts which are sufficient to provide the desired unit dosage.

A proposed daily dose of an active compound of this invention in the combination formulation (a formulation containing an active compound of this invention and a histamine H1 antagonist) for oral, parenteral, rectal or buccal administration to the average adult human for the treatment of the conditions referred to above is from about 0.01 mg to about 2000 mg, preferably from about 0.1 mg to about 200 mg of the active ingredient of formula I per unit dose which could be administered, for example, 1 to 4 times per day.

A proposed daily dose of a histamine H1 antagonist, preferably cetirizine, in the combination formulation for oral, parenteral or buccal administration to the average adult human for the treatment of the conditions referred to above is from about 0.1 mg to about 2000 mg, preferably from about 1 mg to about 200 mg of the histamine H1 antagonist per unit dose which could be administered, for example, 1 to 4 times per day.

A preferred dose ratio of cetirizine to an active compound of this invention in the combination formulation for oral, parenteral or buccal administration to the average adult human for the treatment of the conditions referred to above is from about 0.00005 to about 20,000, preferably from about 0.25 to about 2,000.

Aerosol combination formulations for treatment of the conditions referred to above in the average adult human are preferably arranged so that each metered dose or “puff” of aerosol contains from about 0.01 μg to about 100 mg of the active compound of this invention, preferably from about 1 μg to about 10 mg of such compound. Administration may be several times daily, for example 2, 3, 4 or 8 times, giving for example, 1, 2 or 3 doses each time.

Aerosol formulations for treatment of the conditions referred to above in the average adult human are preferably arranged so that each metered dose or “puff” of aerosol contains from about 0.01 mg to about 2000 mg of a histamine H1 antagonist, preferably cetirizine, preferably from about 1 mg to about 200 mg of cetirizine. Administration may be several times daily, for example 2, 3, 4 or 8 times, giving for example, 1, 2 or 3 doses each time.

As previously indicated, a histamine H1 antagonist, preferably cetirizine, in combination with compounds of formula I are readily adapted to therapeutic use as antiallergy agents. In general, these antiallergy compositions containing a histamine H1 antagonist, preferably cetirizine, and a compound of formula I are normally administered in dosages ranging from about 0.01 mg to about 100 mg per kg of body weight per day of a histamine H1 antagonist, preferably cetirizine, preferably from about 0.1 mg. to about 10 mg per kg of body weight per day of cetirizine; with from about 0.001 mg. to about 100 mg per kg of body weight per day of a compound of formula I, preferably from about 0.01 mg to about 10 mg per kg of body weight per day of a compound of formula I, although variations will necessarily occur depending upon the conditions of the subject being treated and the particular route of administration chosen.

In connection with the use of an active compound of this invention with a 5-HT re-uptake inhibitor, preferably sertraline, for the treatment of subjects possessing any of the above conditions, it is to be noted that these compounds may be administered either alone or in combination with pharmaceutically acceptable carriers by either of the routes previously indicated, and that such administration can be carried out in both single and multiple dosages. More particularly, the active combination can be administered in a wide variety of different dosage forms, i.e., they may be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of formula I are present in such dosage forms at concentration levels ranging from about 0.5% to about 95% by weight of the total composition, i.e., in amounts which are sufficient to provide the desired unit dosage and a 5-HT re-uptake inhibitor, preferably sertraline, is present in such dosage forms at concentration levels ranging from about 0.5% to about 95% by weight of the total composition, i.e., in amounts which are sufficient to provide the desired unit dosage.

A proposed daily dose of an active compound of this invention in the combination formulation (a formulation containing an active compound of this invention and a 5-HT re-uptake inhibitor) for oral, parenteral, rectal or buccal administration to the average adult human for the treatment of the conditions referred to above is from about 0.01 mg to about 2000 mg, preferably from about 0.1 mg to about 200 mg of the active ingredient of formula I per unit dose which could be administered, for example, 1 to 4 times per day.

A proposed daily dose of a 5-HT re-uptake inhibitor, preferably sertraline, in the combination formulation for oral, parenteral or buccal administration to the average adult human for the treatment of the conditions referred to above is from about 0.1 mg to about 2000 mg, preferably from about 1 mg to about 200 mg of the 5-HT re-uptake inhibitor per unit dose which could be administered, for example, 1 to 4 times per day.

A preferred dose ratio of sertraline to an active compound of this invention in the combination formulation for oral, parenteral or buccal administration to the average adult human for the treatment of the conditions referred to above is from about 0.00005 to about 20,000, preferably from about 0.25 to about 2,000.

Aerosol combination formulations for treatment of the conditions referred to above in the average adult human are preferably arranged so that each metered dose or “puff” of aerosol contains from about 0.01 μg to about 100 mg of the active compound of this invention, preferably from about 1 μg to about 10 mg of such compound. Administration may be several times daily, for example 2, 3, 4 or 8 times, giving for example, 1, 2 or 3 doses each time.

Aerosol formulations for treatment of the conditions referred to above in the average adult human are preferably arranged so that each metered dose or “puff” of aerosol contains from about 0.01 mg to about 2000 mg of a 5-HT re-uptake inhibitor, preferably sertraline, preferably from about 1 mg to about 200 mg of sertraline. Administration may be several times daily, for example 2, 3, 4 or 8 times, giving for example, 1, 2 or 3 doses each time.

As previously indicated, a 5-HT re-uptake inhibitor, preferably sertraline, in combination with compounds of formula I are readily adapted to therapeutic use as antidepressant agents. In general, these antidepressant compositions containing a 5-HT re-uptake inhibitor, preferably sertraline, and a compound of formula I are normally administered in dosages ranging from about 0.01 mg to about 100 mg per kg of body weight per day of a 5-HT re-uptake inhibitor, preferably sertraline, preferably from about 0.1 mg. to about 10 mg per kg of body weight per day of sertraline; with from about 0.001 mg. to about 100 mg per kg of body weight per day of a compound of formula I, preferably from about 0.01 mg to about 10 mg per kg of body weight per day of a compound of formula I, although variations will necessarily occur depending upon the conditions of the subject being treated and the particular route of administration chosen.

Anxiety disorders include, for example, generalized anxiety disorder, panic disorder, PTSD, and social anxiety disorder. Mood adjustment disorders include, for example, depressed mood, mixed anxiety and depressed mood, disturbance of conduct, and mixed disturbance of conduct and depressed mood. Attention adjustment disorders include, for example, in addition to ADHD, attention deficit disorders or other cognitive disorders due to general medical conditions. Psychotic disorders include, for example, schizoaffective disorders and schizophrenia; sleep disorders include, for example, narcolepsy and enuresis.

Examples of the disorders or conditions which may be treated by the compound, composition and method of this invention are also as follows: depression, including, for example, depression in cancer patients, depression in Parkinson's patients, post-myocardial Infarction depression, depression in patients with human immunodeficiency virus (HIV), Subsyndromal Symptomatic depression, depression in infertile women, pediatric depression, major depression, single episode depression, recurrent depression, child abuse induced depression, post partum depression, DSM-IV major depression, treatment-refractory major depression, severe depression, psychotic depression, post-stroke depression, neuropathic pain, manic depressive illness, including manic depressive illness with mixed episodes and manic depressive illness with depressive episodes, seasonal affective disorder, bipolar depression BP I, bipolar depression BP II, or major depression with dysthymia; dysthymia; phobias, including, for example, agoraphobia, social phobia or simple phobias; eating disorders, including, for example, anorexia nervosa or bulimia nervosa; chemical dependencies, including, for example, addictions to alcohol, cocaine, amphetamine and other psychostimulants, morphine, heroin and other opioid agonists, phenobarbital and other barbiturates, nicotine, diazepam, benzodiazepines and other psychoactive substances; Parkinson's diseases, including, for example, dementia in Parkinson's disease, neuroleptic-induced parkinsonism or tardive dyskinesias; headache, including, for example, headache associated with vascular disorders; withdrawal syndrome; age-associated learning and mental disorders; apathy; bipolar disorder; chronic fatigue syndrome; chronic or acute stress; conduct disorder; cyclothymic disorder; somatoform disorders such as somatization disorder, conversion disorder, pain disorder, hypochondriasis, body dysmorphic disorder, undifferentiated disorder, and somatoform NOS; incontinence; inhalation disorders; intoxication disorders; mania; oppositional defiant disorder; peripheral neuropathy; post-traumatic stress disorder; late luteal phase dysphoric disorder; specific developmental disorders; SSRI “poop out” syndrome, or a patient's failure to maintain a satisfactory response to SSRI therapy after an initial period of satisfactory response; and tic disorders including Tourette's disease.

As an example, the mammal in need of the treatment or prevention may be a human. As another example, the mammal in need of the treatment or prevention may be a mammal other than a human.

A compound of formula I, which is basic in nature, is capable of forming a wide variety of different salts with various inorganic and organic acids. The acid addition salts are readily prepared by treating the base compounds with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid salts of the active compound used in formulating the pharmaceutical composition of this invention that are basic in nature are those which form non-toxic acid addition salts, i.e., salts containing pharmacologically acceptable anions. Non-limiting examples of the salts include the acetate, benzoate, beta-hydroxybutyrate, bisulfate, bisulfite, bromide, butyne-1,4-dioate, caproate, chloride, chlorobenzoate, citrate, dihydrogenphosphate, dinitrobenzoate, fumarate, glycollate, heptanoate, hexyne-1,6-dioate, hydroxybenzoate, iodide, lactate, maleate, malonate, mandelate, metaphosphate, methanesulfonate, methoxybenzoate, methylbenzoate, monohydrogen phosphate, naphthalene-1-sulfonate, naphthalene-2-sulfonate, oxalate, phenylbutyrate, phenylpropionate, phosphate, phthalate, phenylacetate, propanesulfonate, propiolate, propionate, pyrophosphate, pyrosulfate, sebacate, suberate, succinate, sulfate, sulfite, sulfonate, tartrate, xylenesulfonate, acid phosphate, acid citrate, bitartrate, succinate, gluconate, saccharate, nitrate, methanesulfonate and pamoate [i.e., 1,1′-methylene-bis-(2-hydroxy-3-naphthoate)] salts.

Preferred embodiments of the present invention include the compounds of formula I in which

(A) R¹ and R²together with the nitrogen to which they are attached form a piperidine ring; or

(B) R¹ and R² together with the nitrogen to which they are attached form a pyrrolidine ring; or

(C) embodiment (A) wherein m is three and n is one and R³ and R⁴ are both hydrogen; or R³is methyl and R⁴is hydrogen;

(D) embodiment (B) wherein m is three and n is one and R³ and R⁴ are both hydrogen; or R³is methyl and R⁴ is hydrogen.

The most preferred embodiment of the present invention includes the compounds of formula I in which R¹ and R² together with the nitrogen to which they are attached form a piperidine ring, R³, R⁴ and R⁵ are hydrogen, R⁶ is phenyl, k=0, m=3 and n=1.

Preferred embodiments of the present invention also include any combination of the foregoing embodiments (A)-(D).

Preferred compounds of formula I in accordance with the present invention are the following:
4-[6-(2-piperidin-1-ylethoxy)-1,2,3,4-tetrahydro-naphthalen-2-ylmethyl]-morpholine;
1-methyl-4-[6-(2-piperidin-1-ylethoxy)-1,2,3,4-tetrahydro-naphthalen-2-ylmethyl]-piperazine;
1-methyl-4-{1-[6-(2-piperidin-1-ylethoxy)-1,2,3,4-tetrahydro-naphthalen-2-yl]-ethyl}-piperazine;
tert-butyl-{1-[6-(2-piperidin-1-ylethoxy)-1,2,3,4-tetrahydro-naphthalen-2-yl]-ethyl}-methylamine;
1-[2-(5-methyl-6-pyrrolidin-1-ylmethyl-5,6,7,8-tetrahydro-naphthalen-2-yloxy)-ethyl]-piperidine;
1-[2-(6-pyrrolidin-1-ylmethyl-5,6,7,8-tetrahydro-naphthalen-2-yloxy)-ethyl]-pyrrolidine;
1-[2-(6-(2,5-dimethyl)pyrrolidin-1-ylmethyl-5,6,7,8-tetrahydro-naphthalen-2-yloxy)-ethyl]-pyrrolidine;
N-benzyl-[2-(6-pyrrolidin-1-ylmethyl-5,6,7,8-tetrahydro-naphthalen-2-yloxy)-ethyl]-methylamine;
1-[2-(3-methyl-6-pyrrolidin-1-ylmethyl-5,6,7,8-tetrahydro-naphthalen-2-yloxy)-ethyl]-piperidine;
1-[2-(1,3-difluoro-6-pyrrolidin-1-ylmethyl-5,6,7,8-tetrahydro-naphthalen-2-yloxy)-ethyl]-piperidine;
1-[2-(1-fluoro-6-pyrrolidin-1-ylmethyl-5,6,7,8-tetrahydro-naphthalen-2-yloxy)-ethyl]-piperidine;
1-[2-(2-pyrrolidin-1-ylmethylindan-5-yloxy)-ethyl]-piperidine;
1-[3-(2-pyrrolidin-1-ylmethyl-indan-5-yloxy)-propyl]-pyrrolidine;
2-[3-(6-pyrrolidin-1-ylmethyl-5,6,7,8-tetrahydro-naphthalen-2-yloxy)-propyl]-octahydro-isoindole;
2-[3-(6-pyrrolidin-1-ylmethyl-5,6,7,8-tetrahydro-naphthalen-2-yloxy)-propyl]-octahydro-cyclopenta[c] pyrrole;
2-methyl-5-[3-(6-pyrrolidin-1-ylmethyl-5,6,7,8-tetrahydro-naphthalen-2-yloxy)-propyl]-octahydro-pyrrolo[3,4-c]pyrrole;
pyridin-4-yl-[3-(6-pyrrolidin-1-ylmethyl-5,6,7,8-tetrahydro-naphthalen-2-yloxy)-propyl]-methylamine;
pyrimidin-2-yl-[3-(6-pyrrolidin-1-ylmethyl-5,6,7,8-tetrahydro-naphthalen-2-yloxy)-propyl]-methylamine;
isopropyl-[3-(6-pyrrolidin-1-ylmethyl-5,6,7,8-tetrahydro-naphthalen-2-yloxy)-propyl]-phenylamine;
cyclopentyl-[3-(6-pyrrolidin-1-ylmethyl-5,6,7,8-tetrahydro-naphthalen-2-yloxy)-propyl]-isopropylamine;
2,5-dimethyl-1-[3-(6-pyrrolidin-1-ylmethyl-5,6,7,8-tetrahydro-naphthalen-2-yloxy)-propyl]-pyrrolidine;
1-[4-(6-pyrrolidin-1-ylmethyl-5,6,7,8-tetrahydro-naphthalen-2-yloxy)-butyl]-pyrrolidine;
1-[3-(6-pyrrolidin-1-ylmethyl-6,7,8,9-tetrahydro-5H-benzocyclohepten-2-yloxy)-propyl]-piperidine;
1-[2-(6-pyrrolidin-1-ylmethyl-6,7,8,9-tetrahydro-5H-benzocyclohepten-2-yloxy)-ethyl]-piperidine;
1-[2-(3,3-dimethyl-2-pyrrolidin-1-ylmethylindan-5-yloxy)-ethyl]-piperidine;
1-[4-(2-pyrrolidin-1-ylmethylindan-5-yloxy)-butyl]-piperidine;
1-[2,2-dimethyl-3-(2-pyrrolidin-1-ylmethylindan-5-yloxy)-propyl]-piperidine;
1-{2-methyl-1-[2-(2-pyrrolidin-1-ylmethylindan-5-yloxy)-ethyl]-propyl}-piperidine;
1-{3-[2-(1-pyrrolidin-1-yl-ethyl)-indan-5-yloxy]-propyl}-piperidine;
4-{1-[5-(3-piperidin-1-ylpropoxy)-indan-2-yl]-ethyl}-thiomorpholine;
4-{1-[5-(3-piperidin-1-ylpropoxy)-indan-2-yl]-ethyl}-thiomorpholine 1-oxide;
4-{1-[5-(3-piperidin-1-ylpropoxy)-indan-2-yl]-ethyl}-thiomorpholine 1,1-dioxide;
N-isopropyl-[5-(3-piperidin-1-ylpropoxy)-indan-2-ylmethyl]-methylamine;
1-[2-(5-methyl-6-pyrrolidin-1-ylmethyl-5,6,7,8-tetrahydro-naphthalen-2-yloxy)-ethyl]-piperidine;
1-[3-(6-methyl-6-pyrrolidin-1-ylmethyl-5,6,7,8-tetrahydro-naphthalen-2-yloxy)-propyl]-piperidine;
N-pyridin-2-yl-[2-(6-pyrrolidin-1-ylmethyl-5,6,7,8-tetrahydro-naphthalen-2-yloxy)-ethyl]-methylamine;
pyrimidin-4-yl-[2-(6-pyrrolidin-1-ylmethyl-5,6,7,8-tetrahydro-naphthalen-2-yloxy)-ethyl]-amine;
1-[3-(6-pyrrolidin-1-ylmethyl-5,6,7,8-tetrahydro-naphthalen-2-yloxy)-propyl]-1,2,3,4-tetrahydro-quinoline;
7-chloro-2-[3-(6-pyrrolidin-1-ylmethyl-5,6,7,8-tetrahydro-naphthalen-2-yloxy)-propyl]-1,2,3,4-tetrahydro-isoquinoline;
2-[3-(3-fluoro-6-pyrrolidin-1-ylmethyl-5,6,7,8-tetrahydro-naphthalen-2-yloxy)-propyl]-5-methoxy-2,3-dihydro-1H-isoindole;
{6-[3-(5,7-dihydropyrrolo[3,4-b]pyridin-6-yl)-propoxy]-1,2,3,4-tetrahydro-naphthalen-2-ylmethyl}-dimethylamine;
6-[2-(6-pyrrolidin-1-ylmethyl-5,6,7,8-tetrahydro-naphthalen-2-yloxy)-ethyl]-5,6,7,8-tetrahydro-[1,6]naphthyridine;
3-[2-(6-pyrrolidin-1-ylmethyl-5,6,7,8-tetrahydro-naphthalen-2-yloxy)-ethyl]-3-azabicyclo[3.3.1]nonane;
7-[2-(6-pyrrolidin-1-ylmethyl-5,6,7,8-tetrahydro-naphthalen-2-yloxy)-ethyl]-7-azabicyclo[2.2.1]heptane; and
1-{6-[2-(2-phenylaziridin-1-yl)-ethoxy]-1,2,3,4-tetrahydro-naphthalen-2-ylmethyl}-pyrrolidine.
The most preferred examples of compounds according to the present invention include:
(±)-[3-(6-pyrrolidin-1-ylmethyl-5,6,7,8-tetrahydro-naphthalen-2-yloxy)-propyl]-dimethylamine hydrochloride;
(±)-dimethyl-[2-(6-pyrrolidin-1-ylmethyl-5,6,7,8-tetrahydro-naphthalen-2-yloxy)-ethyl]-amine hydrochloride;
(±)-1-[3-(6-pyrrolidin-1-ylmethyl-5,6,7,8-tetrahydro-naphthalen-2-yloxy)-propyl]-piperidine;
(±)4-[3-(6-pyrrolidin-1-ylmethyl-5,6,7,8-tetrahydro-naphthalen-2-yloxy)-propyl]-morpholine hydrochloride;
(−)-4-[3-(6-pyrrolidin-1-ylmethyl-5,6,7,8-tetrahydro-naphthalen-2-yloxy)-propyl]-morpholine hydrochloride;
(±)-1-[3-(6-pyrrolidin-1-ylmethyl-5,6,7,8-tetrahydro-naphthalen-2-yloxy)-propyl]-pyrrolidine hydrochloride;
(±)-1′-[3-(6-pyrrolidin-1-ylmethyl-5,6,7,8-tetrahydro-naphthalen-2-yloxy)-propyl]-[1,4′]bi-piperidinyl hydrochloride;
(±)-1-[3-(6-pyrrolidin-1-ylmethyl-5,6,7,8-tetrahydro-naphthalen-2-yloxy)-propyl]-piperidine-4-carboxylic acid amide hydrochloride;
(±)-1-[3-(6-pyrrolidin-1-ylmethyl-5,6,7,8-tetrahydro-naphthalen-2-yloxy)-propyl]-piperidine-3-carboxylic acid amide;
(−)-1-[3-(6-pyrrolidin-1-ylmethyl-5,6,7,8-tetrahydro-naphthalen-2-yloxy)-propyl]-piperidine hydrochloride;
(−)-4-[3-(6-pyrrolidin-1-ylmethyl-5,6,7,8-tetrahydro-naphthalen-2-yloxy)-propyl]-morpholine hydrochloride;
(+)-1-[3-(6-pyrrolidin-1-ylmethyl-5,6,7,8-tetrahydro-naphthalen-2-yloxy)-propyl]-piperidine hydrochloride;
(−)-[3-(6-pyrrolidin-1-ylmethyl-5,6,7,8-tetrahydro-naphthalen-2-yloxy)-propyl]-dimethylamine hydrochloride;
(+)-4-[3-(6-pyrrolidin-1-ylmethyl-5,6,7,8-tetrahydro-naphthalen-2-yloxy)-propyl]-morpholine hydrochloride;
(−)-3,3-dimethyl-1-[3-(6-pyrrolidin-1-ylmethyl-5,6,7,8-tetrahydro-naphthalen-2-yloxy)-propyl]-piperidine hydrochloride;
(−)-[3-(6-pyrrolidin-1-ylmethyl-5,6,7,8-tetrahydro-naphthalen-2-yloxy)-propyl]-spiro[5.5]undec-3-ylamine hydrochloride;
(+)-bicyclo[2.2.1]hept-2-yl-[3-(6-pyrrolidin-1-ylmethyl-5,6,7,8-tetrahydro-naphthalen-2-yloxy)-propyl]-amine hydrochloride;
(+)-3-[3-(6-pyrrolidin-1-ylmethyl-5,6,7,8-tetrahydro-naphthalen-2-yloxy)-propyl]-3-azaspiro-[5.5]undecane hydrochloride;
(−)-cyclopropyl-[3-(6-pyrrolidin-1-ylmethyl-5,6,7,8-tetrahydro-naphthalen-2-yloxy)-propyl]-amine;
(−)-1-{4-phenyl-1-[3-(6-pyrrolidin-1-ylmethyl-5,6,7,8-tetrahydro-naphthalen-2-yloxy)-propyl]-piperidin-4-yl}-ethanone;
(−)-(1,2-dimethylpropyl)-[3-(6-pyrrolidin-1-ylmethyl-5,6,7,8-tetrahydro-naphthalen-2-yloxy)-propyl]-amine;
(+)-1-{3-[3-(6-pyrrolidin-1-ylmethyl-5,6,7,8-tetrahydro-naphthalen-2-yloxy)-propylamino]-propyl}-pyrrolidin-2-one;
(−)-1-{3-[3-(6-pyrrolidin-1-ylmethyl-5,6,7,8-tetrahydro-naphthalen-2-yloxy)-propylamino]-propyl}-pyrrolidin-2-one;
(+)-N,N-dimethyl-N′-[3-(6-pyrrolidin-1-ylmethyl-5,6,7,8-tetrahydro-naphthalen-2-yloxy)-propyl]-ethane-1,2-diamine;
(+)-(4-chlorobenzyl)-[3-(6-pyrrolidin-1-ylmethyl-5,6,7,8-tetrahydro-naphthalen-2-yloxy)-propyl]-amine;
(−)-(4-chlorobenzyl)-[3-(6-pyrrolidin-1-ylmethyl-5,6,7,8-tetrahydro-naphthalen-2-yloxy)-propyl]-amine;
(−)-2-{ethyl-[3-(6-pyrrolidin-1-ylmethyl-5,6,7,8-tetrahydro-naphthalen-2-yloxy)-propyl]-amino}-ethanol;
(+)-{1-[3-(6-pyrrolidin-1-ylmethyl-5,6,7,8-tetrahydro-naphthalen-2-yloxy)-propyl]-piperidin-3-yl}-methanol;
(+)-(3,4-dichlorobenzyl)-[3-(6-pyrrolidin-1-ylmethyl-5,6,7,8-tetrahydro-naphthalen-2-yloxy)-propyl]-amine;
(−)-(3,4-dichlorobenzyl)-[3-(6-pyrrolidin-1-ylmethyl-5,6,7,8-tetrahydro-naphthalen-2-yloxy)-propyl]-amine;
(+)-benzyl-[3-(6-pyrrolidin-1-ylmethyl-5,6,7,8-tetrahydro-naphthalen-2-yloxy)-propyl]-amine;
(−)-benzyl-[3-(6-pyrrolidin-1-ylmethyl-5,6,7,8-tetrahydro-naphthalen-2-yloxy)-propyl]-amine;
(+)-(2-pyridin-2-ylethyl)-[3-(6-pyrrolidin-1-ylmethyl-5,6,7,8-tetrahydro-naphthalen-2-yloxy)-propyl]-amine;
(−)-(2-pyridin-2-ylethyl)-[3-(6-pyrrolidin-1-ylmethyl-5,6,7,8-tetrahydro-naphthalen-2-yloxy)-propyl]-amine;
(+)-(4-tert-butylcyclohexyl)-[3-(6-pyrrolidin-1-ylmethyl-5,6,7,8-tetrahydro-naphthalen-2-yloxy)-propyl]-amine;
(−)-(4-tert-butylcyclohexyl)-[3-(6-pyrrolidin-1-ylmethyl-5,6,7,8-tetrahydro-naphthalen-2-yloxy)-propyl]-amine;
(+)-(3-methoxybenzyl)-[3-(6-pyrrolidin-1-ylmethyl-5,6,7,8-tetrahydro-naphthalen-2-yloxy)-propyl]-amine;
(−)-(3-methoxybenzyl)-[3-(6-pyrrolidin-1-ylmethyl-5,6,7,8-tetrahydro-naphthalen-2-yloxy)-propyl]-amine;
(+)-(4-fluorobenzyl)-[3-(6-pyrrolidin-1-ylmethyl-5,6,7,8-tetrahydro-naphthalen-2-yloxy)-propyl]-amine;
(−)-(4-fluorobenzyl)-[3-(6-pyrrolidin-1-ylmethyl-5,6,7,8-tetrahydro-naphthalen-2-yloxy)-propyl]-amine;
(+)-(2,6-difluorobenzyl)-[3-(6-pyrrolidin-1-ylmethyl-5,6,7,8-tetrahydro-naphthalen-2-yloxy)-propyl]-amine;
(−)-(2,6-difluorobenzyl)-[3-(6-pyrrolidin-1-ylmethyl-5,6,7,8-tetrahydro-naphthalen-2-yloxy)-propyl]-amine;
(+)-(3-ethoxypropyl)-[3-(6-pyrrolidin-1-ylmethyl-5,6,7,8-tetrahydro-naphthalen-2-yloxy)-propyl]-amine;
(−)-(3-ethoxypropyl)-[3-(6-pyrrolidin-1-ylmethyl-5,6,7,8-tetrahydro-naphthalen-2-yloxy)-propyl]-amine;
(+)-[3-(6-pyrrolidin-1-ylmethyl-5,6,7,8-tetrahydro-naphthalen-2-yloxy)-propyl]-N-cyclohexyl-N-methylamine;
(−)-[3-(6-pyrrolidin-1-ylmethyl-5,6,7,8-tetrahydro-naphthalen-2-yloxy)-propyl]-N-cyclohexyl-N-methylamine;
(+)-(2,2-dimethylpropyl)-[3-(6-pyrrolidin-1-ylmethyl-5,6,7,8-tetrahydro-naphthalen-2-yloxy)-propyl]-amine;
(−)-(2,2-dimethylpropyl)-[3-(6-pyrrolidin-1-ylmethyl-5,6,7,8-tetrahydro-naphthalen-2-yloxy)-propyl]-amine;
(+)-1-cyclohexyl-4-[3-(6-pyrrolidin-1-ylmethyl-5,6,7,8-tetrahydro-naphthalen-2-yloxy)-propyl]-piperazine;
(−)-1-cyclohexyl-4-[3-(6-pyrrolidin-1-ylmethyl-5,6,7,8-tetrahydro-naphthalen-2-yloxy)-propyl]-piperazine;
(+)-1-[3-(6-pyrrolidin-1-ylmethyl-5,6,7,8-tetrahydro-naphthalen-2-yloxy)-propyl]-piperidine-4-carboxylic acid amide;
(−)-1-[3-(6-pyrrolidin-1-ylmethyl-5,6,7,8-tetrahydro-naphthalen-2-yloxy)-propyl]-piperidine-4-carboxylic acid amide;
(+)-1-{4-[3-(6-pyrrolidin-1-ylmethyl-5,6,7,8-tetrahydro-naphthalen-2-yloxy)-propyl]-piperazin-1-yl}-ethanone;
(−)-1-{4-[3-(6-pyrrolidin-1-ylmethyl-5,6,7,8-tetrahydro-naphthalen-2-yloxy)-propyl]-piperazin-1-yl}-ethanone;
(+)-3-methyl-1-[3-(6-pyrrolidin-1-ylmethyl-5,6,7,8-tetrahydro-naphthalen-2-yloxy)-propyl]-piperidine;
(−)-3-methyl-1-[3-(6-pyrrolidin-1-ylmethyl-5,6,7,8-tetrahydro-naphthalen-2-yloxy)-propyl]-piperidine;
(+)-2-methyl-1-[3-(6-pyrrolidin-1-ylmethyl-5,6,7,8-tetrahydro-naphthalen-2-yloxy)-propyl]-piperidine;
(−)-2-methyl-1-[3-(6-pyrrolidin-1-ylmethyl-5,6,7,8-tetrahydro-naphthalen-2-yloxy)-propyl]-piperidine;
(+)-4-methyl-1-[3-(6-pyrrolidin-1-ylmethyl-5,6,7,8-tetrahydro-naphthalen-2-yloxy)-propyl]-piperidine;
(−)-4-methyl-1-[3-(6-pyrrolidin-1-ylmethyl-5,6,7,8-tetrahydro-naphthalen-2-yloxy)-propyl]-piperidine;
(+)-{1-[3-(6-pyrrolidin-1-ylmethyl-5,6,7,8-tetrahydro-naphthalen-2-yloxy)-propyl]-pyrrolidin-3-yl}-diethylamine;
(−)-{1-[3-(p-Pyrrolidin-1-ylmethyl-5,6,7,8-tetrahydro-naphthalen-2-yloxy)-propyl]-pyrrolidin-3-yl}-diethylamine;
(+)-methyl-(1-methylpiperidin-4-yl)-[3-(6-pyrrolidin-1-ylmethyl-5,6,7,8-tetrahydro-naphthalen-2-yloxy)-propyl]-amine;
(−)-methyl-(1-methylpiperidin-4-yl)-[3-(6-pyrrolidin-1-ylmethyl-5,6,7,8-tetrahydro-naphthalen-2-yloxy)-propyl]-amine;
(+)-1-(3,5-dichloropyridin-4-yl)-4-[3-(6-pyrrolidin-1-ylmethyl-5,6,7,8-tetrahydro-naphthalen-2-yloxy)-propyl]-piperazine;
(−)-1-(3,5-dichloropyridin-4-yl)-4-[3-(6-pyrrolidin-1-ylmethyl-5,6,7,8-tetrahydro-naphthalen-2-yloxy)-propyl]-piperazine;
(+)-2-{4-[3-(6-pyrrolidin-1-ylmethyl-5,6,7,8-tetrahydro-naphthalen-2-yloxy)-propyl]-piperazin-1-yl}-pyrimidine;
(−)-2-{4-[3-(6-pyrrolidin-1-ylmethyl-5,6,7,8-tetrahydro-naphthalen-2-yloxy)-propyl]-piperazin-1-yl}-pyrimidine;
(+)-2,2,N,N-tetramethyl-N′-[3-(6-pyrrolidin-1-ylmethyl-5,6,7,8-tetrahydro-naphthalen-2-yloxy)-propyl]-propane-1,3-diamine;
(−)-2,2,N,N-tetramethyl-N′-[3-(6-pyrrolidin-1-ylmethyl-5,6,7,8-tetrahydro-naphthalen-2-yloxy)-propyl]-propane-1,3-diamine;
(+)-2-[3-(6-pyrrolidin-1-ylmethyl-5,6,7,8-tetrahydro-naphthalen-2-yloxy)-propyl]-2,3-dihydro-1H-isoindole;
(−)-2-[3-(6-pyrrolidin-1-ylmethyl-5,6,7,8-tetrahydro-naphthalen-2-yloxy)-propyl]-2,3-dihydro-1H-isoindole;
(+)-{1-[3-(6-pyrrolidin-1-ylmethyl-5,6,7,8-tetrahydro-naphthalen-2-yloxy)-propylamino]-cyclopentyl}-methanol;
(−)-{1-[3-(6-pyrrolidin-1-ylmethyl-5,6,7,8-tetrahydro-naphthalen-2-yloxy)-propylamino]-cyclopentyl}-methanol;
(+)-2,6-dimethyl-4-[3-(6-pyrrolidin-1-ylmethyl-5,6,7,8-tetrahydro-naphthalen-2-yloxy)-propyl]-morpholine;
(−)-2,6-dimethyl-4-[3-(6-pyrrolidin-1-ylmethyl-5,6,7,8-tetrahydro-naphthalen-2-yloxy)-propyl]-morpholine;
(+)-(1-methyl-2-morpholin-4-ylethyl)-[3-(6-pyrrolidin-1-ylmethyl-5,6,7,8-tetrahydro-naphthalen-2-yloxy)-propyl]-amine;
(−)-(1-methyl-2-morpholin-4-ylethyl )-[3-(6-pyrrolidin-1-ylmethyl-5,6,7,8-tetrahydro-naphthalen-2-yloxy)-propyl]-amine;
(+)-cyclohexylmethyl-[3-(6-pyrrolidin-1-ylmethyl-5,6,7,8-tetrahydro-naphthalen-2-yloxy)-propyl]-amine;
(−)-cyclohexylmethyl-[3-(6-pyrrolidin-1-ylmethyl-5,6,7,8-tetrahydro-naphthalen-2-yloxy)-propyl]-amine;
(+)-(4-morpholin-4-yl-tetrahydrofuran-3-yl)-[3-(6-pyrrolidin-1-ylmethyl-5,6,7,8-tetrahydro-naphthalen-2-yloxy)-propyl]-amine;
(−)-(4-morpholin-4-yl-tetrahydrofuran-3-yl )-[3-(6-pyrrolidin-1-ylmethyl-5,6,7,8-tetrahydro-naphthalen-2-yloxy)-propyl]-amine;
(+)-(1-methyl-1-phenylethyl)-[3-(6-pyrrolidin-1-ylmethyl-5,6,7,8-tetrahydro-naphthalen-2-yloxy)-propyl]-amine;
(−)-(1-methyl-1-phenylethyl )-[3-(6-pyrrolidin-1-ylmethyl-5,6,7,8-tetrahydro-naphthalen-2-yloxy)-propyl]-amine;
(+)-furan-2-ylmethyl-[3-(6-pyrrolidin-1-ylmethyl-5,6,7,8-tetrahydro-naphthalen-2-yloxy)-propyl]-methylamine;
(−)-furan-2-ylmethyl-[3-(6-pyrrolidin-1-ylmethyl-5,6,7,8-tetrahydro-naphthalen-2-yloxy)-propyl]-methylamine;
(+)-furan-2-ylmethyl-[3-(6-pyrrolidin-1-ylmethyl-5,6,7,8-tetrahydro-naphthalen-2-yloxy)-propyl]-amine;
(−)-furan-2-ylmethyl-[3-(6-pyrrolidin-1-ylmethyl-5,6,7,8-tetrahydro-naphthalen-2-yloxy)-propyl]-amine;
(+)-2-{1-[3-(6-pyrrolidin-1-ylmethyl-5,6,7,8-tetrahydro-naphthalen-2-yloxy)-propyl]-pyrrolidin-3-yl}-pyridine;
(−)-2-{1-[3-(6-pyrrolidin-1-ylmethyl-5,6,7,8-tetrahydro-naphthalen-2-yloxy)-propyl]-pyrrolidin-3-yl}-pyridine;
(+)-[3-(6-pyrroldin-1-ylmethyl-5,6,7,8-tetrahydro-naphthalen-2-yloxy)-propyl]-(tetrahydro-pyran-4-ylmethyl)-amine;
(−)-[3-(6-pyrrolidin-1-ylmethyl-5,6,7,8-tetrahydro-naphthalen-2-yloxy)-propyl]-(tetrahydro-pyran-4-ylmethyl )-amine;
(+)-2-{1-[3-(6-pyrrolidin-1-ylmethyl-5,6,7,8-tetrahydro-naphthalen-2-yloxy)-propyl]-pyrrolidin-2-yl}-pyridine;
(−)-2-{1-[3-(6-pyrrolidin-1-ylmethyl-5,6,7,8-tetrahydro-naphthalen-2-yloxy)-propyl]-pyrrolidin-2-yl}-pyridine;
(+)-(1-isopropyl-piperidin-4-ylmethyl)-[3-(6-pyrrolidin-1-ylmethyl-5,6,7,8-tetrahydro-naphthalen-2-yloxy)-propyl]-amine;
(−)-(1-isopropyl-piperidin-4-ylmethyl)-[3-(6-pyrrolidin-1-ylmethyl-5,6,7,8-tetrahydro-naphthalen-2-yloxy)-propyl]-amine;
(+)-[2-(4-fluorophenyl)-ethyl]-[3-(6-pyrrolidin-1-ylmethyl-5,6,7,8-tetrahydro-naphthalen-2-yloxy)-propyl]-amine;
(−)-[2-(4-fluorophenyl)-ethyl]-[3-(6-pyrrolidin-1-ylmethyl-5,6,7,8-tetrahydro-naphthalen-2-yloxy)-propyl]-amine;
(+)-2-methyl-6-[3-(6-pyrrolidin-1-ylmethyl-5,6,7,8-tetrahydro-naphthalen-2-yloxy)-propylamino]-heptan-2-ol;
(−)-2-methyl-6-[3-(6-pyrrolidin-1-ylmethyl-5,6,7,8-tetrahydro-naphthalen-2-yloxy)-propylamino]-heptan-2-ol;
(+)-(1-isopropyl-piperidin-3-ylmethyl)-[3-(6-pyrrolidin-1-ylmethyl-5,6,7,8-tetrahydro-naphthalen-2-yloxy)-propyl]-amine;
(−)-(1-isopropyl-piperidin-3-ylmethyl)-[3-(6-pyrrolidin-1-ylmethyl-5,6,7,8-tetrahydro-naphthalen-2-yloxy)-propyl]-amine;
(+)-[3-(6-pyrrolidin-1-ylmethyl-5,6,7,8-tetrahydro-naphthalen-2-yloxy)-propyl]-cyclohexyl-amine;
(−)-[3-(6-pyrrolidin-1-ylmethyl-5,6,7,8-tetrahydro-naphthalen-2-yloxy)-propyl]-cyclohexyl-amine;
(+)-6-[3-(6-pyrrolidin-1-ylmethyl-5,6,7,8-tetrahydro-naphthalen-2-yloxy)-propyl]-6-azabicyclo-[3.2.1]octane;
(−)-6-[3-(6-pyrrolidin-1-ylmethyl-5,6,7,8-tetrahydro-naphthalen-2-yloxy)-propyl]-6-azabicyclo-[3.2.1]octane;
(+)-4-methoxy-1-[3-(6-pyrrolidin-1-ylmethyl-5,6,7,8-tetrahydro-naphthalen-2-yloxy)-propyl]-piperidine;
(−)-4-methoxy-1-[3-(6-pyrrolidin-1-ylmethyl-5,6,7,8-tetrahydro-naphthalen-2-yloxy)-propyl]-piperidine;
(+)-(2-{1-[3-(6-pyrrolidin-1-ylmethyl-5,6,7,8-tetrahydro-naphthalen-2-yloxy)-propyl]-piperidin-4-yl}-ethyl)-dimethylamine;
(−)-(2-{1-[3-(6-pyrrolidin-1-ylmethyl-5,6,7,8-tetrahydro-naphthalen-2-yloxy)-propyl]-piperidin-4-yl}-ethyl)-dimethylamine;
(+)-2,6-dimethyl-4-{1-[3-(6-pyrrolidin-1-ylmethyl-5,6,7,8-tetrahydro-naphthalen-2-yloxy)-propyl]-piperidin-4-yl}-morpholine;
(−)-2,6-dimethyl-4-{1-[3-(6-pyrrolidin-1-ylmethyl-5,6,7,8-tetrahydro-naphthalen-2-yloxy)-propyl]-piperidin-b 4-yl}-morpholine;
(+)-2-[3-(6-pyrrolidin-1-ylmethyl-5,6,7,8-tetrahydro-naphthalen-2-yloxy)-propyl]-octahydro-pyrrolo[1,2-a]pyrazine;
(−)-2-[3-(6-pyrrolidin-1-ylmethyl-5,6,7,8-tetrahydro-naphthalen-2-yloxy)-propyl]-octahydro-pyrrolo[1,2-a]pyrazine;
(+)-(1H-imidazol-2-ylmethyl)-methyl-[3-(6-pyrrolidin-1-ylmethyl-5,6,7,8-tetrahydro-naphthalen-2-yloxy)-propyl]-amine;
(−)-(1H-imidazol-2-ylmethyl)-methyl-[3-(6-pyrrolidin-1-ylmethyl-5,6,7,8-tetrahydro-naphthalen-2-yloxy)-propyl]-amine;
(+)-2-(4-fluorophenyl)-1-[3-(6-pyrrolidin-1-ylmethyl-5,6,7-8-tetrahydro-naphthalen-2-yloxy)-propyl]-pyrrolidine;
(−)-2-(4-fluorophenyl)-1-[3-(6-pyrrolidin-1-ylmethyl-5,6,7-8-tetrahydro-naphthalen-2-yloxy)-propyl]-pyrrolidine;
(+)-(2-{1-[3-(6-pyrrolidin-1-ylmethyl-5,6,7,8-tetrahydro-naphthalen-2-yloxy)-propyl]-piperidin-4-yloxy}-ethyl)-dimethylamine;
(−)-(2-{1-[3-(6-pyrrolidin-1-ylmethyl-5,6,7,8-tetrahydro-naphthalen-2-yloxy)-propyl]-piperidin-4-yloxy}-ethyl)-dimethylamine;
(−)-(2-pyrrolidin-1-ylethyl)-[3-(6-pyrrolidin-1-ylmethyl-5,6,7,8-tetrahydro-naphthalen-2-yloxy)-propyl]-amine;
(−)-(2-chlorobenzyl)-[3-(6-pyrrolidin-1-ylmethyl-5,6,7,8-tetrahydro-naphthalen-2-yloxy)-propyl]-amine;
(−)-pyridin-3-ylmethyl-[3-(6-pyrrolidin-1-ylmethyl-5,6,7,8-tetrahydro-naphthalen-2-yloxy)-propyl]-amine;
(−)-(2-piperidin-1-ylethyl)-[3-(6-pyrrolidin-1-ylmethyl-5,6,7,8-tetrahydro-naphthalen-2-yloxy)-propyl]-amine;
(−)-(3,4-difluorobenzyl)-[3-(6-pyrrolidin-1-ylmethyl-5,6,7,8-tetrahydro-naphthalen-2-yloxy)-propyl]-amine;
(−)-N,N,N′-trimethyl-N′-[3-(6-pyrrolidin-1-ylmethyl-5,6,7,8-tetrahydro-naphthalen-2-yloxy)-propyl]-ethane-1,2-diamine;
(−)-(2,4-dichlorobenzyl)-[3-(6-pyrrolidin-1-ylmethyl-5,6,7,8-tetrahydro-naphthalen-2-yloxy)-propyl]-amine;
(−)-(3-imidazol-1-yl-propyl)-[3-(6-pyrrolidin-1-ylmethyl-5,6,7,8-tetrahydro-naphthalen-2-yloxy)-propyl]-amine;
(−)-3,5-dimethyl-1-[3-(6-pyrrolidin-1-ylmethyl-5,6,7,8-tetrahydro-naphthalen-2-yloxy)-propyl]-piperidine;
(−)-1-pyridin-2-yl-4-[3-(6-pyrrolidin-1-ylmethyl-5,6,7,8-tetrahydro-naphthalen-2-yloxy)-propyl]-piperazine;
(−)-(3-azepan-1-ylpropyl)-[3-(6-pyrrolidin-1-ylmethyl-5,6,7,8-tetrahydro-naphthalen-2-yloxy)-propyl]-amine;
(−)-1-[3-(6-pyrrolidin-1-ylmethyl-5,6,7,8-tetrahydro-naphthalen-2-yloxy)-propyl]-1,2,3,4,5,6-hexahydro-[4,4′]bipyridinyl;
(−)-furan-3-ylmethyl-[3-(6-pyrrolidin-1-ylmethyl-5,6,7,8-tetrahydro-naphthalen-2-yloxy)-propyl]-amine;
(−)-1,6-dimethyl-2-[3-(6-pyrrolidin-1-ylmethyl-5,6,7,8-tetrahydro-naphthalen-2-yloxy)-propyl]-1,2,3,4-tetrahydro-pyrrolo[1,2-a]pyrazine;
(−)-[3-(6-pyrrolidin-1-ylmethyl-5,6,7,8-tetrahydro-naphthalen-2-yloxy)-propyl]-[2-(tetrahydro-pyran-2-yl)-ethyl]-amine;
(−)-(2-imidazol-1-yl-ethyl)-[3-(6-pyrrolidin-1-ylmethyl-5,6,7,8-tetrahydro-naphthalen-2-yloxy)-propyl]-amine;
(−)-(1H-imidazol-2-ylmethyl)-[3-(6-pyrrolidin-1-ylmethyl-5,6,7,8-tetrahydro-naphthalen-2-yloxy)-propyl]-amine;
(−)-[3-(6-pyrrolidin-1-ylmethyl-5,6,7,8-tetrahydro-naphthalen-2-yloxy)-propyl]-(tetrahydro-pyran-4-ylmethyl)-methylamine; and
(−)-1-[2-(6-pyrrolidin-1-ylmethyl-5,6,7,8-tetrahydro-naphthalen-2-yloxy)-ethyl]-piperidine dihydrochloride.

### DETAILED DESCRIPTION OF THE INVENTION

The compounds of formula I according to the invention may be prepared by the general procedure shown in Scheme 1-3.

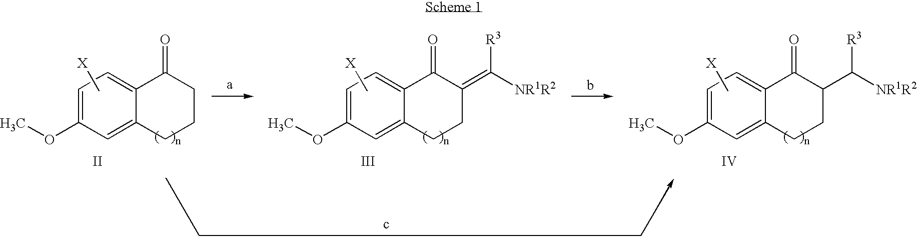

According to Scheme 1, a ketone of the general formula II can be reacted with an amide acetal of the general formula (IX):
R³—C(OR¹⁰)₂NR¹R² (IX)
wherein OR¹⁰ is a lower alkoxy group or, together with the carbon to which they are attached form a 5-8 member cyclic ring, to form the keto-enamine intermediate of the general formula III (step a). Similar procedures have been described by N. Dereu and M. Renson, Journal of Organometallic Chemistry (1981) 208:11; R. Gammill, Synthesis (1979) 901; B. Fohlisch, Chemische Berichte (1971) 104:348. Preparation of the amide acetals IX is well precedented in the chemical literature, e.g., H. Elingsfeld, M. Seefelder, H. Weidinger, (Badische Anilin- & Soda-Fabrik A.-G.) DE-1119872 (1961); V. Virmani, J. Singh, P. Jain, N. Anand, Journal of the Chemical Society of Pakistan (1979) 1(2):109-15; H. Moehrle, H. Dwuletzki, Chemische Berichte (1986) 119(12):3591-9; H. Bredereck, G. Simchen, S. Rebsdat, W. Kantlehner, P. Horn, R. Wahl, H. Hoffmann, P. Grieshaber, Chemische Berichte (1968) 101(1):41-50.

When the acetal IX is not available, an alternative procedure as described by A. Kreutzberger and E. Kreutzberger, Tetrahedron (1976) 32:2603 in which the ketone II and a secondary amine of general formula HNR¹R² are reacted in the presence of 1,3,5-triazine to give intermediate III, may be employed. The intermediates III so prepared may be isolated as the pure Z- or E-isomers, or as a mixture of both geometric isomer forms.

Alternatively, compounds of general formula II may first be reacted with an orthoester of the general formula R³(OR¹²)₃, wherein OR¹² is typically OCH₃ or OC₂H₅, to produce an intermediate of general formula XIV:

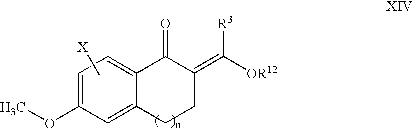

This intermediate may then be reacted with an appropriate amine of the general formula HNR¹R² to give an intermediate of general formula III. This process is analogous to one disclosed by Howard in U.S. Pat. No. 4,476,307 (Oct. 9, 1984)

The intermediates of formula III may then be converted to the compounds of general formula IV using one or more available reagents that are selective for reducing a C═C double bond (step b). Such procedures include: treating the intermediate of formula III with lithium aluminum hydride (LiAlH₄) in a reaction inert solvent such as THF or diethyl ether (ref. J. P. Michael, A. S. Parsons and R. Hunter, Tetrahedron Letters (1989) 30:4879; P. F. Schuda, C. B. Ebner and T. M. Morgan, Tetrahedron Letters (1989) 27(23):2567); reaction of III with magnesium metal in ethanol (ref. R. Brettle and S. M. Shibib, Journal of the Chemical Society—Part I (1981) 2912-2919); hydrogenation using platinum (II) oxide in methanol (ref. P. Kloss, Chemische Berichte (1964) 97:1723) or sodium bis-(2-methoxyethoxy)aluminum hydride in ethanol or benzene (ref. V. W. Weselowski and A. M. Moiseenkov, Synthesis (1974) 58).

Alternatively, a ketone of the general formula II and an amine of general formula HNR¹R² can be reacted under conditions described in the chemical literature to produce an intermediate of the general formula IV directly—this reaction is referred to as the Mannich reaction and generally applies to the use of secondary amines, i.e., where R¹ and R² are not hydrogen (step c); for a review, see M. Tramontoni and L. Angiolini, Tetrahedron (1990) 46(6): 1791-1837. In this reaction, the ketone (II) and the amine are combined in the presence of a reactive aldehyde (R³CHO) under acidic conditions until the reaction is judged to be complete. The addition of silver metal has been reported to facilitate this reaction (ref. S. J. Joglekar and S. D. Samant, Synthesis (1988) 830).

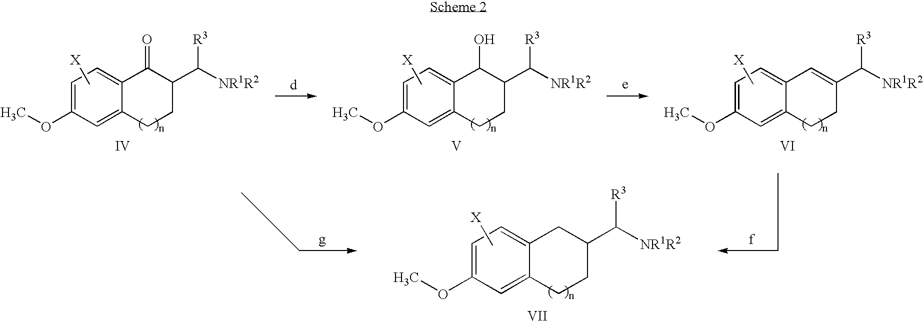

The carbonyl group in the intermediate IV can then be removed in one or more steps to produce the intermediate of formula VII, as shown in Scheme 2. For example, the ketone IV can be reduced to the corresponding alcohol of formula V by treatment with reducing agents such as sodium borohydride (NaBH₄), lithium borohydride (LiBH₄), lithium aluminum hydride (LiAlH₄) and the like (step d). Conditions for such reductions have been described in the chemical literature, e.g., M. Hudlicky in “Reductions in Organic Chemistry”, Ellis Horwood: Chichester, UK (1984), 96 and R. C. Larock in “Comprehensive Organic Transformations”. VCH: New York, N.Y. (1989), 527.

The alcohol intermediate V so obtained may be converted to the corresponding alkene of general formula VI through the elimination of water to form the C═C double bond (step e). This dehydration step can be achieved in high yield using one or more procedures well known to those skilled in the art. For example, treating the alcohol V with a strong mineral acid like hydrochloric or sulfuric acid, or heating the alcohol in the presence of p-toluenesulfonic acid or activated molecular sieves in a reaction inert solvent (e.g., toluene) can produce the intermediate VI. For further review, see R. C. Larock, ibid, 151.

The alkene intermediate of general formula VI may then be converted to the intermediate of formula VII by reduction of the C═C double bond (step f). This has been accomplished using reduction conditions known and reported in the chemical literature. For example, hydrogenation using hydrogen (H₂) gas, in the presence of a catalyst like palladium on carbon (Pd/C) and in a reaction inert solvent such as methanol or ethanol at one to three atmospheres of pressure can produce the intermediate VII. It can also be appreciated that such reductions of the alkene bond may produce a mixture of the R- and S-isomers; however, selective catalysts can be employed to favor production of one or the other of these isomers. The alkene group in the intermediate VII may also reduced by the use of reagents such as formic acid in the presence of a catalyst like Pd/C, or samarium (II) iodide among many others. An in-depth review of the hydrogenation of alkene carbon-carbon double bonds can be found in M. B. Smith and J. March, “March's Advanced Organic Chemistry”, 5^{th} Ed., J. Wiley & Sons, Inc., New York (2001) 1002-1007 and the references listed therein.

In some instances, the dehydration step e and the reduction step f (i.e., conversion of V to VII) can be performed in a single step, for example by the addition of a proportional amount of hydrochloric acid to the hydrogenation mixture to generate the alkene in situ.

Alternatively, the reduction of the intermediate ketone of general formula IV to the intermediate alkane of general formula VII may be achieved directly (step g). The chemical literature includes many examples of such conversions, including the use of a zinc-mercury amalgam under acidic conditions, referred to as the Clemmensen reduction of ketones (ref. R. Ghosh, R. Robinson, Journal of the Chemical Society (1944) 506). These reaction conditions may not be suitable for the reduction of intermediates containing acid sensitive groups, e.g., nitrites. Similarly, the ketone may be reduced to the corresponding methylene intermediate VII via the Wolff-Kischner reduction, by first preparing a hydrazone of the general formula X (e.g., R¹¹═H) followed by decomposition with a strong base. Examples of such reductions have been described by R. Schwarz and K. Capek, Monatsch. Chemie (1952) 83:883; R. Hirschmann and W. S. Johnson, Journal of the American Chemical Society (1951) 73:326.

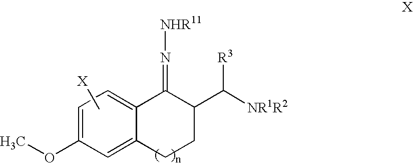

A variation that may be used effectively is the reduction of a tosylhydrazone X (wherein R¹¹ is 4-methylphenylsulfonyl) that may be accomplished using reagents like NaBH₄ or LiAlH₄ under conditions described previously to produce the intermediate VII. Additional references may be found in Smith and March, ibid, 1547-1549.

Reduction of the aryl ketone of intermediate IV may also be accomplished using ammonium formate and 10% palladium on carbon (ref. S. Ram and L. D. Spicer, Tetrahedron Letters (1988) 29:3741), hydrogenation with H₂ in the presence of Pd/C catalyst and perchloric acid (J. G. Cannon, J. P. Pease and J. Flynn, Journal of Medicinal Chemistry (1984) 27(7):922; C. Grethe et al, Journal of Organic Chemistry (1968) 33:494), or lithium aluminum hydride—aluminum chloride in tetrahydrofuran (J. G. Cannon et al, Journal of Medicinal Chemistry (1984) 27(2):190.

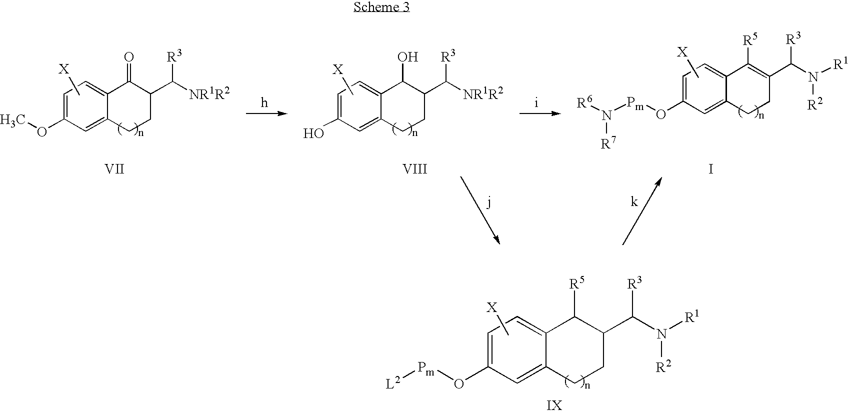

The methyl ether of the intermediate of general formula VII may then be converted to the hydroxyl group of general formula VIII, as depicted in Scheme 3 (step h). Thus, the intermediate VII can be heated in the presence of hydrobromic acid in acetic acid (ref. D. Dhut, J. P. Glay and J. Barbe, Synthetic Communications (1991) 21:969), or reacted with aqueous HBr under conditions described by D. Ramesh et al, Journal of Organic Chemistry (1988) 53(1):212 to remove the methyl group. Other procedures to produce the intermediates of general formula VIII will include reacting VII with boron tribromide in dichloromethane (M. Hori et al, Chemical and Pharmaceutical Bulletin of Japan (1990) 38(1): 8; J. Martin and C. J. Moody, Journal of the Chemical Society, Part I (1988) (2):235; J. Kleinschroth and J. Hartenstein, Synthesis (1988) 970); boron trichloride (R. S. Michalak et al, Tetrahedron Letters (1989) 30:4783; C. F. Carvalho and M. V. Sargent, Journal of the Chemical Society—Chemical Communications (1984) 227); boron trichloride—dimethylsulfide complex (V. H. Rawal and M. P. Cava, Journal of the American Chemical Society (1986) 108:2110); boron trifluoride diethyl etherate in dichlormethane (V. O. Omuaru and W. B. Whalley, Indian Journal of Chemistry (1989) 28:1009); sodium thiomethylate in DMF (L. Testaferri et al, Synthesis (1983) 751); tetrachlorsilane and sodium iodide in toluene-acetonitrile (C. C. Kanakam et al, Journal of the Chemical Society—Perkin Transformations I (1989) 1907); and trimethylsilyl iodide (S. Gunzenhauser and H. Balli, Helvetica Chimica Acta (1985) 68:56).

The phenol intermediate of formula VIII can then be reacted with a reagent of the general formula XI:
L¹—Pₘ—NR⁶R⁷ (XI)
wherein the group L¹ is an independent leaving group (e.g., Cl, Br, iodine, mesylate, tosylate and the like) to produce the desired compounds of general formula I (step i). Compounds of general formula XI are commercially available or readily prepared using methods described in the chemical literature.

An alternative to this last step is provided by the method outlined in Scheme 3. Thus, reaction of the intermediate hydroxyl compound of general formula VIII with a compound of the general formula XII:
L¹—Pₘ—L² (XII)
wherein L¹ is as defined above and L² is a suitable leaving group like those defined for L¹, may be used to prepare an intermediate of general formula IX (step j). Reaction of the intermediate of formula IX with an amine of formula HNR¹R² in a reaction inert solvent may produce the title compounds of the general formula I (step k). In this situation, it may be desirable to select the leaving groups such that L¹ is more reactive than L²; alternatively, an excess of the compound XII can be employed to minimize the formation of XIII by the subsequent reaction intermediate IX with a second molecule of VIII, i.e.:

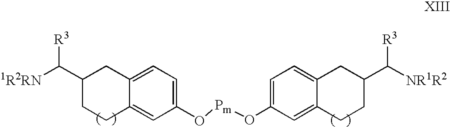

In the examples that follow, the abbreviations used are intended to have the following, general meaning:

bm: broad multiplet (NMR)

bs: broad singlet (NMR)

dd: doublet of doublets (NMR)

d.e.: diatomaceous earth, filter agent

DMF: dimethyformamide

LRMS: low resolution mass spectrometry

calcd; calculated

d; doublet (NMR)

EtOAc: ethyl acetate

J: coupling constant (NMR)

LAH: lithium aluminum hydride

m: multiplet (in NMR)

min: minute(s)

m/z: mass to charge ratio (in mass spectrometry)

obsd: observed

Rf: retention factor (in chromatography)

Rt: retention time (in chromatography)

rt: room temperature

s: singlet (NMR), second(s)

t: triplet

THF: tetrahydrofuran

tlc: thin layer chromatography

Solvents were purchased and used without purification. Yields were calculated for material judged homogenous by thin layer chromatography and NMR. Thin layer chromatography was performed on Merck Kieselgel 60 F 254 plates eluting with the solvents indicated, visualized by a 254 nm UV lamp, and stained with either an aqueous KMnO₄ solution or an ethanolic solution of 12-molybdophosphoric acid. Flash column chromatography was performed with using either pre-packed Biotage® or ISCO® columns using the size indicated. Nuclear magnetic resonance (NMR) spectra were acquired on a Unity 400 or 500 at 400 MHz or 500 MHz for ¹H, respectively, and 100 MHz or 125 MHz for ¹³C NMR, respectively. Chemical shifts for proton ¹H NMR spectra are reported in parts per million relative to the singlet of CDCl₃ at 7.24 ppm. Chemical shifts for ¹³C NMR spectra are reported in parts per million downfield relative to the centerline of the triplet of CDCl₃ at 77.0 ppm. Mass spectra analyses were performed on a APCI Gilson 215, micromass ZMD (50% Acetonitrile/50% water) spectrometer.

Reactions under microwave conditions were done using 2-5 mL round bottom vials, fitted with septa. The vials containing the reactants were inserted into the reaction chamber of a EMRYS™ Creator microwave apparatus (maximum power of 300 W) from Personal Chemistry Inc., 25 Birch St., Bldg C, Suite 304, Milford, Mass. 01757 and heated to the appropriate temperature for a the prescribed period of time. HPLC was performed according to the following methods:

Method A: Preparative conditions (Waters 600 & Waters 2767 Sample Manager); Column: Waters Symmetry C₁₈, 5 μm, 30×150 mm steel column, part # WAT248000, serial # M12921A01; solvent A—0.1% Trifluoroacetic acid/water; solvent B—Acetonitrile; volume of injection: 850 μL; time 0.0, 100% solvent A, 0% solvent B, flow 20; time 2.0, 100% solvent A, 0% solvent B, flow 20; time 12.0, 0% solvent A, 100% solvent B, flow 20; time 15.0, 0% solvent A, 100% solvent B, flow 20; time 15.1, 100% solvent A, 0% solvent B, flow 20; time 20.0, 100% solvent A, 0% solvent B, flow 20.

Mass spectral (micromassZO) conditions; Capillary(kV): 3.0; Cone (V): 20; Extractor (V): 3.0; RF Lens (V): 0.5; Source temp. (° C.): 120; Desolvation temp. (° C.): 360; Desolvation gas flow (L/hr): 450; Cone gas flow (L/hr): 150; LM Resolution: 15; HM Resolution: 15; Ion Energy: 0.2; Multiplier: 550.

Splitter; Acurate by LC Packings, 1/10,000; Upchurch needle valve setting: 14; Make up pump (Waters 515) Flow (ml/min.): 1. PDA (Waters 996) Settings; Start/End wavelength (nm): 200/600; Resolution: 1.2; Sample Rate: 1; Channels: TIC, 254 nm and 220 nm.

Method B: Preparative conditions (Waters 600 & Waters 2767 Sample Manager); Column: Waters Xterra PrepMS C₁₈ column, 5 μm, 30×150 mm steel column, part # 186001120, serial # T22881T 09; solvent A—0.1% Trifluoroacetic acid/water; solvent B—Acetonitrile; volume of injection: 1050 μL; time 0.0, 100% solvent A, 0% solvent B, flow 20; time 2.0, 100% solvent A, 0% solvent B, flow 20; time 12.0, 0% solvent A, 100% solvent B, flow 20; time 14.0, 0% solvent A, 100% solvent B, flow 20; time 14.1, 100% solvent A, 0% solvent B, flow 20; time 19.1, 100% solvent A, 0% solvent B, flow 20.

Mass spectral (micromassZO) conditions; Capillary(kV): 3.0; Cone (V): 20; Extractor (V): 3.0; RF Lens (V): 0.5; Source temp. (° C.): 120; Desolvation temp. (° C.): 360; Desolvation gas flow (L/hr): 450; Cone gas flow (L/hr): 150; LM Resolution: 15; HM Resolution: 15; Ion Energy: 0.2; Multiplier: 550.

Splitter; Acurate by LC Packings, 1/10,000; Upchurch needle valve setting: 14; Make up pump (Waters 515) Flow (ml/min.): 1. PDA (Waters 996) Settings; Start/End wavelength (nm): 200/600; Resolution: 1.2; Sample Rate: 1; Channels: TIC, 254 nm and 220 nm.

Method C: Preparative conditions (Waters 600 & Waters 2767 Sample Manager); Column: Waters Symmetry C₁₈, 5 μm, 30×150 mm steel column, part # WAT248000, serial # M12921A01; solvent A—0.1% Trifluoroacetic acid/water; solvent B—Acetonitrile; volume of injection: 850 μL; time 0.0, 90% solvent A, 10% solvent B, flow 20; time 10.0, 0% solvent A, 100% solvent B, flow 20; time 12.0, 0% solvent A, 100% solvent B, flow 20.

Mass spectral (micromassZO) conditions; Capillary(kV): 3.0; Cone (V): 20; Extractor (V): 3.0; RF Lens (V): 0.5; Source temp. (° C.): 120; Desolvation temp. (° C.): 360; Desolvation gas flow (L/hr): 450; Cone gas flow (L/hr): 150; LM Resolution: 15; HM Resolution: 15; Ion Energy: 0.2; Multiplier: 550. Splitter; Acurate by LC Packings, 1/10,000; Upchurch needle valve setting: 14; Make up pump (Waters 515) Flow (ml/min.): 1. PDA (Waters 996) Settings; Start/End wavelength (nm): 200/600; Resolution: 1.2; Sample Rate: 1; Channels: TIC, 254 nm and 220 nm.

The following intermediates may be prepared by the procedures described above:

### INTERMEDIATE 1

### 6-Methoxy-2-pyrrolidin-1-ylmethyl-3,4-dihydro-2H-naphthalen-1-one

Prepared in 86% yield from 6-methoxy-1-tetralone (100 g, Aldrich Chemical Co.), pyrrolidine (42.3 g, Aldrich) and paraformaldehyde (56 g) according to the method of W. Welch, U.S. Pat. No. 4,022,791 (May 10, 1977).

M.p. (Hydrochloride salt) 187.0-187.4° C. (ref: 190-191° C.).

Mass spectrum (m/z) calcd for C₁₆H₂₁NO₂: 259.35; obsd. 260 (M+1).

### INTERMEDIATE 2

### 6-Methoxy-2-pyrrolidin-1-ylmethyl-1,2,3,4-tetrahydro-naphthalen-1-ol

Prepared in 98% yield from 33.15 g of intermediate 1 in 500 mL of anhydrous THF at 0° C. treated with 256 mL of 1.0 molar LiAlH₄ in THF. The product was isolated as a pale yellow oil, 32.95 g.

¹H-nmr (CDCl₃, 400 MHz) δ 1.40 (m, 1H), 1.65-1.68 (m, 1H), 1.70-1.88 (m, 4H), 2.45 (d, 1H), 2.48-2.55 (m, 2H), 2.62-2.80 (m, 3H), 2.84-2.95 (m, 2H), 3.75 (s, 3H), 4.62 (d, 1H), 6.58 (m, 1H), 6.77 (d, 1H), 7.40 (bs, 1H), 7.55 (m, 1H).

Mass spectrum (m/z) calcd for C₁₆H₂₃NO₂: 261.36; obsd. 262 (M+1).

### INTERMEDIATE 3

### 1-(6-Methoxy-3,4-dihydro-naphthalen-2-ylmethyl)-pyrrolidine

Prepared according to the method of Welch, intermediate 2 (38 g) in 1 L water was treated with 500 mL of 48% aqueous hydrobromic acid to produce, after workup, intermediate 3 as a light brown oil, 23.6 g (67%).

¹H-nmr (DMSO-d₆, 400 MHz) δ 1.85 (m, 2H), 1.97 (m, 2H), 2.30 (t, 2H), 2.74 (dd, 2H), 3.03 (m, 2H), 3.44 (m, 2H), 3.70 (s, 3H), 3.84 (d, 2H), 6.60 (s, 1H), 6.75 (m, 2H), 7.03 (d, 1H), 9.65 (bs, HBr).

Mass spectrum (m/z) calcd for C₁₆H₂₁NO: 243.35; obsd 244 (M+1).

### INTERMEDIATE 4

### (±)-1-(6-Methoxy-1,2,3,4-tetrahydro-naphthalen-2-ylmethyl)-pyrrolidine

A mixture of 1-(6-methoxy-3,4-dihydronaphthalen-2-ylmethyl)-pyrrolidine (23.6 g, 97 mmol) and 6N HCl (50 mL) in 700 mL of 30% aqueous ethanol was cautiously added, under a nitrogen atmosphere, to a 2.0 L Parr bottle containing 4.0 g of 10% palladium on carbon. The mixture was hydrogenated on a Parr shaker apparatus for 5 hr at an initial pressure of 45 psi. The mixture was filtered through a pad of d.e., the pad was washed with additional aqueous ethanol and the filtrates were combined, concentrated in vacuo to a volume of about 400 mL, diluted with dichloromethane and water and the treated with 6N NaOH until the aqueous layer was basic (pH>10.0). The organic layer was removed and combined with additional CH₂Cl₂ extractions of the aqueous layer. The combined organic layers were washed with H₂O, then saturated aqueous NaCl and dried over MgSO₄. Removal of the solvent in vacuo gave a tan oil, 20.11 g (84%)

¹H-nmr (CDCl₃, 400 MHz) δ 1.36 (m, 1H), 1.78 (m, 4H), 1.92 (m, 2H), 2.32-2.55 (m, 7H), 2.78 (m, 2H), 2.86 (dd, 1H), 3.74 (s, 3H), 6.62 (s, 1H), 6.67 (dd, 1H), 7.00 (d, 1H).

Mass spectrum (m/z) calcd for C₁₆H₂₃NO: 245.36; obsd. 246 (M+1).

Using a chiral chromatography method, the racemate was separated into the individual enantiomers:

### (+)-1-(6-methoxy-1,2,3,4-tetrahydro-naphthalen-2-ylmethyl)-pyrrolidine

[α]²⁵_{D}=+72.0° (c=0.91, CHCl₃);

### (−)-1-(6-methoxy-1,2,3,4-tetrahydro-naphthalen-2-ylmethyl)-pyrrolidine

[α]²⁵_{D}=−84.6° (c=1.52, CDCl₃).

### INTERMEDIATE 5

### (+)-6-Pyrrolidin-1-ylmethyl-5,6,7,8-tetrahydro-naphthalen-2-ol

A solution of (+)-1-(6-methoxy-1,2,3,4-tetrahydro-naphthalen-2-ylmethyl)-pyrrolidine (7.3 g, 29.3 mol, intermediate 4) and 38 mL of 48% hydrogen bromide in 140 mL of acetic acid was heated under N₂ at 90-95° C. for 24 hr, cooled to rt and concentrated in vacuo to a waxy amber solid. Water and ethyl acetate were added to dissolve the solid and concentrated NH₄OH was added to adjust the pH to >9.0. After stirring the mixture for 30 min, the EtOAc layer was removed and combined with additional EtOAc extractions of the aqueous layer. The organic layers were washed with H₂O, then saturated NaCl, and finally dried with MgSO₄ before concentrating in vacuo to an amber-colored oil, 6.59 g. The oil slowly solidified on standing to a solid, m.p. 120.0-120.8° C.

¹H-nmr (CDCl₃, 400 MHz) δ 1.68 (m, 1H), 1.80 (bs, 4H), 1.95 (bm, 2H), 2.30-2.43 (m, 4H), 2.50 (bs, 4H), 2.75 (m, 2H), 2.85 (d, 1H), 6.58 (dd, 2H), 6.85 (d, 1H).

Mass spectrum (m/z) calcd for C₁₅H₂₁NO: 231.34; obsd. 232 (M+1).

(−)-6-pyrrolidin-1-ylmethyl-5,6,7,8-tetrahydro-naphthalen-2-ol was prepared in the same manner to produce a pale brown oil, 6.44 g (89%). This oil (231 mg) was converted with 1.0 M HCl in Et₂O to the hydrochloride salt, 225 mg. M.p. 224.5-225.0° C.

Alternatively, 5.7 g (23.23 mmol) of the (−) methyl ether (intermediate 4) in 170 mL of dichloromethane was treated at 0° C. with 1.0 M boron tribromide for 18 hr to give, after aqueous workup, a tan residue, 5.86 g.

(±)-6-pyrrolidin-1-ylmethyl-5,6,7,8-tetrahydro-naphthalen-2-ol hydrobromide was similarly prepared in 96% yield as a tan solid, 7.6 g.

¹H-nmr (CDCl₃, 400 MHz) δ 1.61 (m, 1H), 2.10 (bm, 3H), 2.29 (bm, 3H), 2.58 (m, 1H), 2.83 (m, 2H), 2.88 (m, 1H), 3.27 (bm, 2H), 3.75 (q, 2H), 4.24 (bs, 2H), 4.58 (bs, 1H), 6.56 (m, 1H), 6.61 (dd, 1H), 6.92 (d, 1H).

### INTERMEDIATE 6

### (−)-1-[6-(3-Chloropropoxy)-1,2,3,4-tetrahydro-naphthalen-2-ylmethyl]-pyrrolidine

A mixture of (−)-6-pyrrolidin-1-ylmethyl-5,6,7,8-tetrahydro-naphthalen-2-ol (12.8 g, 39.0 mmol) in 100 mL anhydrous THF was stirred at rt while 1.0 M potassium tert-butoxide in THF (68 mL, 68 mmol) was added via syringe. After an additional 45 min, 3-bromo-1-chloropropane (6.7 g, 43 mmol) was added and the mixture was heated to reflux for 18 hr, at which time additional 3-bromo-1-chlorpropane (0.68 mL) was added. After a further 5 hr at reflux, the reaction was cooled to rt, the tan slurry was diluted with water and the dark organic layer was removed and combined with an EtOAc extract of the aqueous layer. The combined organic layers were washed with water, then saturated NaCl, and dried over MgSO₄. Concentration in vacuo gave the crude product as a brown oil, 13.1 g. Chromatography on silica gel, eluting with CDCl₃, then 5% CH₃OH in CDCl₃ gave the purified product as a viscous oil, 7.9 g.

¹H-nmr (CDCl₃, 400 MHz) δ 1.61 (m, 2H), 2.05-2.21 (m, 4H), 2.27-2.35 (m, 4H), 2.60 (m, 1H), 2.82 (m, 4H), 3.05 (m, 1H), 3.18 (m, 1H), 3.75 (s, 2H), 3.97 (m, 1H), 4.07 (m, 2H), 4.22 (m, 1H), 6.65 (m, 1H), 6.73 (m, 1H), 7.02 (m, 1H).

Mass spectrum (m/z) calcd for C₁₈H₂₆ClNO: 307.87; obsd. 308 (M+1), 310.

In the same manner, 11.3 g of (+)-6-pyrrolidin-1-ylmethyl-5,6,7,8-tetrahydro-naphthalen-2-ol was converted to (+)-1-[6-(3-chloropropoxy)-1,2,3,4-tetrahydro-naphthalen-2-ylmethyl]-pyrrolidine.

### EXAMPLE 1

### General procedure A

### (±)-[3-(6-Pyrrolidin-1-ylmethyl-5,6,7,8-tetrahydro-naphthalen-2-yloxy)-propyl]-dimethylamine hydrochloride

A mixture of (±)-6-pyrrolidin-1-ylmethyl-5,6,7,8-tetrahydro-naphthalen-2-ol hydrobromide (0.328 g, 1.66 mmol, intermediate 5) and sodium hydride (0.218 g of a 60% oil dispersion, 5.46 mmol) in 10 mL of anhydrous DMF was stirred at rt for 15 min, then treated with dimethylaminopropyl chloride hydrochloride (0.289 g, 1.83 mmol, Aldrich Chemical Co.) and stirred overnight at rt. The mixture was diluted with water and EtOAc, separated and the aqueous layer re-extracted with additional EtOAc. The organic layers were combined and extracted with cold 1N HCl, then cold water. The acidic aqueous extracts were combined, made basic with 2N NaOH and then extracted once more with portions of EtOAc. The organic extracts were washed with H₂O, then saturated NaCl and dried over MgSO₄. After filtering, the solvent was removed in vacuo to give a light brown oil, 0.225 g. The free base was dissolved in diethyl ether and treated with 1N HCl in Et₂O (Aldrich Chemical Co.), stirred, filtered and dried to a pale tan solid, 0.123 g. M.P. 187.7-188.7° C.

Mass spectrum (m/z) calcd for C₂₀H₃₂N₂O: 316.48; obsd. 317 (M+1).

¹H-nmr (CDCl₃, 400 MHz) δ 1.30 (m, 1H), 1.55 (m, 1H), 2.06 (m, 6H), 2.84 (m, 6H), 2.86 (m, 1H), 2.96 (m, 1H), 3.12 (m, 2H), 3.24 (s, 6H), 3.30 (m, 1H), 3.53 (m, 2H), 3.95 (bt, 2H), 6.65 (m, 2H), 6.95 (dd, 1H).

The following compounds were also prepared using the general procedure A, as described for Example1:

### EXAMPLE 2

### (±)-Dimethyl-[2-(6-pyrrolidin-1-ylmethyl-5,6,7,8-tetrahydro-naphthalen-2-yloxy)-ethyl]-amine hydrochloride

M.p. 131.6-132.8° C.

Mass spectrum (m/z) calcd for C₁₉H₃₀N₂O: 302.46; obsd. 303 (M+1).

### EXAMPLE 3

### (±)-1-[3-(6-Pyrrolidin-1-ylmethyl-5,6,7,8-tetrahydro-naphthalen-2-yloxy)-propyl]-piperidine

M.p. 180.0-181.4° C.

Mass spectrum (m/z) calcd for C₂₃H₃₆N₂O: 356.55; obsd. 357 (M+1).

¹H-nmr (CDCl₃, 400 MHz) δ 1.45 (bm, 2H), 1.64 (m, 5H), 2.04 (m, 5H), 2.31 (m, 3H), 2.53 (m, 6H), 2.84 (m, 2H), 2.91 (m, 1H), 3.31 (m, 2H), 3.74 (dq, 2H), 3.95 (t, 2H), 4.23 (bs, 2H), 6.59 (d, 1H), 6.67 (dd, 1H), 6.94 (d, 1H).

### EXAMPLE 4

### (±)-4-[3-(6-Pyrrolidin-1-ylmethyl-5,6,7,8-tetrahydro-naphthalen-2-yloxy)-propyl]-morpholine hydrochloride

M.p. 259.9-261.4° C.

Mass spectrum (m/z) calcd for C₂₂H₃₄N₂O: 358.52; obsd. 359 (M+1).

¹H-nmr (DMSO-d₆, 400 MHz) δ 1.34 (m, 2H), 1.90 (m, 3H), 2.11 (m, 3H), 2.36 (dd, 1H), 2.71 (m, 1H), 2.84 (dd, 1H), 3.06 (m, 6H), 3.16 (m, 2H), 3.31 (s, 4H), 3.38 (dd, 2H), 3.53 (m, 2H), 3.77 (t, 2H), 3.92 (m, 1H), 3.95 (m, 2H), 6.62 (bs, 1H), 6.65 (dd, 1H), 6.92 (d, 1H).

### EXAMPLE 5

### (−)-4-[3-(6-Pyrrolidin-1-ylmethyl-5,6,7,8-tetrahydro-naphthalen-2-yloxy)-propyl]-morpholine hydrochloride

M.p. 255.4-256.4° C.

Mass spectrum (m/z) calcd for C₂₂H₃₄N₂O₂: 358.52; obsd. 359 (M+1).

### EXAMPLE 6

### General Procedure B

### (±)-1-[3-(6-Pyrrolidin-1-ylmethyl-5,6,7,8-tetrahydro-naphthalen-2-yloxy)-propyl]-pyrrolidine hydrochloride

Under N₂, a mixture of (±)-1-[6-(3-chloropropoxy)-1,2,3,4-tetrahydro-naphthalen-2-ylmethyl]-pyrrolidine (0.71 g, 2.31 mmol, the title product of intermediate 6), pyrrolidine (0.423 mL, 0.361 g, 5.0 mmol), potassium carbonate (0.957 g, 6.93 mmol) and potassium iodide (70 mg) in 25 mL of methyl ethyl ketone was heated at reflux for 24 hr. After cooling to rt, the mixture was diluted with H₂O and extracted several times with EtOAc. The combined organic layers were washed with H₂O, then saturated NaCl. After drying with MgSO₄, the solvent was removed in vacuo to provide an amber colored oil, 1.24 g. This was chromatographed on silica gel (40×70 mm column) eluting with 5% CH₃OH in CDCl3, the product fractions concentrated in vacuo to a pale brown oil, 135 mg. The oil was dissolved in Et₂O and treated with 1 N HCl in Et₂O, stirred and concentrated to dryness. The residue was treated with a minimal amount of EtOAc and stirred overnight to produce light tan solids that were filtered, washed with Et₂O and dried.

M.p. 149.4-150.5° C.

Mass spectrum (m/z) calcd for C₂₂H₃₄N₂O: 342.52; obsd. 343 (M+1).

¹H-nmr (DMSO-d₆, 400 MHz) δ 1.35 (m, 1H), 1.55 (m, 1H), 1.95 (m, 5H), 2.08 (m, 4H), 2.71 (m, 5H), 3.07 (m, 1H), 3.18 (m, 1H), 3.30 (s, 2H), 3.59 (m, 5H), 3.73 (t, 1H), 3.95 (m, 2H), 4.46 (m, 1H), 6.64 (m, 2H), 6.95 (m, 1H).

The following compounds were also prepared using the general procedure B, as described for Example 6:

### EXAMPLE 7

### (±)-1′-[3-(6-Pyrrolidin-1-ylmethyl-5,6,7,8-tetrahydro-naphthalen-2-yloxy)-propyl]-[1,4′]bi-piperidinyl hydrochloride

M.p. 284.8° C. (decomp.)

Mass spectrum (m/z) calcd for C₂₈H₄₅N₃O: 439.68; obsd. 440 (M+1).

### EXAMPLE 8

### (±)-1-[3-(6-Pyrrolidin-1-ylmethyl-5,6,7,8-tetrahydro-naphthalen-2-yloxy)-propyl]-piperidine-4-carboxylic acid amide hydrochloride

M.p. 211.0-213.0° C.

Mass spectrum (m/z) calcd for C₂₄H₃₇N₃O₂: 399.57; obsd. 400 (M+1).

### EXAMPLE 9

### (±)-1-[3-(6-Pyrrolidin-1-ylmethyl-5,6,7,8-tetrahydro-naphthalen-2-yloxy)-propyl]-piperidine-3-carboxylic acid amide

M.p. 102.0-103.2° C.

Mass spectrum (m/z) calcd for C₂₄H₃₇N₃O₂: 399.57; obsd. 400 (M+1).

### EXAMPLE 10

### (−)-1-[3-(6-Pyrrolidin-1-ylmethyl-5,6,7,8-tetrahydro-naphthalen-2-yloxy)-propyl]-piperidine hydrochloride

[α]^{D}₂₅=−56.98° (c=1.03, CH₃OH)

M.p. 286.6-286.8° C.

Mass spectrum (m/z) calcd for C₂₃H₃₆N₂O: 356.55; obsd. 357 (M+1).

### EXAMPLE 11

### (−)4-[3-(6-Pyrrolidin-1-ylmethyl-5,6,7,8-tetrahydro-naphthalen-2-yloxy)-propyl]-morpholine hydrochloride

M.p. 281.8-282.7° C.

Mass spectrum (m/z) calcd for C₂₂H₃₄N₂O₂: 358.52; obsd. 359 (M+1).

### EXAMPLE 12

### (+)-1-[3-(6-Pyrrolidin-1-ylmethyl-5,6,7,8-tetrahydro-naphthalen-2-yloxy)-propyl]-piperidine hydrochloride

[α]^{D}₂₅=+46.9° (c=1.17, CH₃OH)

M.p. 256.6-258.4° C.

Mass spectrum (m/z) calcd for C₂₃H₃₆N₂O: 356.55; obsd. 357 (M+1).

### EXAMPLE 13

### (−)-[3-(6-Pyrrolidin-1-ylmethyl-5,6,7,8-tetrahydro-naphthalen-2-yloxy)-propyl]-dimethylamine hydrochloride

M.p. 230.6-232.8° C.

Mass spectrum (m/z) calcd for C₂₀H₃₂N₂O: 316.49; obsd. 317 (M+1).

### EXAMPLE 14

### (+)-4-[3-(6-Pyrrolidin-1-ylmethyl-5,6,7,8-tetrahydro-naphthalen-2-yloxy)-propyl]-morpholine hydrochloride

[α]^{D}₂₅=+43.67° (c=0.90, CH₃OH)

M.p. 249.6-250.4° C.

Mass spectrum (m/z) calcd for C₂₂H₃₄N₂O₂: 358.52; obsd. 359 (M+1).

### EXAMPLE 15

### (−)-3,3-Dimethyl-1-[3-(6-pyrrolidin-1-ylmethyl-5,6,7,8-tetrahydro-naphthalen-2-yloxy)-propyl]-piperidine hydrochloride

M.p. 202.0-204.0° C.

Mass spectrum (m/z) calcd for C₂₅H₄₀N₂O: 384.6; obsd. 385 (M+1).

### EXAMPLE 16

### (−)-[3-(6-Pyrrolidin-1-ylmethyl-5,6,7,8-tetrahydro-naphthalen-2-yloxy)-propyl]-spiro[5.5]undec-3-ylamine hydrochloride

M.p. 291.5-292.9° C.

Mass spectrum (m/z) calcd for C₂₉H₄₆N₂O: 438.7; obsd. 439 (M+1).

### EXAMPLE 17

### (+)-Bicyclo[2.2.1]hept-2-yl-[3-(6-pyrrolidin-1-ylmethyl-5,6,7,8-tetrahydro-naphthalen-2-yloxy)-propyl]-amine hydrochloride

M.p. 265.4-266.3° C.

Mass spectrum (m/z) calcd for C₂₅H₃₈N₂O: 382.37; obsd. 383 (M+1).

### EXAMPLE 18

### (+)-3-[3-(6-Pyrrolidin-1-ylmethyl-5,6,7,8-tetrahydro-naphthalen-2-yloxy)-propyl]-3-azaspiro-[5.5]undecane hydrochloride

M.p. 287.6-290.2° C.

Mass spectrum (m/z) calcd for C₂₈H₄₄N₂O: 424.67; obsd. 425 (M+1).

### EXAMPLE 19

### (−)-Cyclopropyl-[3-(6-pyrrolidin-1-ylmethyl-5,6,7,8-tetrahydro-naphthalen-2-yloxy)-propyl]-amine

Mass spectrum (m/z) calcd for C₂₁H₃₂N₂O: 328.5; obsd. 329 (M+1).

### EXAMPLE 20

### (−)-1-{4-Phenyl-1-[3-(6-pyrrolidin-1-ylmethyl-5,6,7,8-tetrahydro-naphthalen-2-yloxy)-propyl]-piperidin-4-yl}-ethanone

Mass spectrum (m/z) calcd for C₃₁H₄₂N₂O₂: 474.69; obsd. 475 (M+1).

### EXAMPLE 21

### (−)-(1,2-Dimethylpropyl)-[3-(6-pyrrolidin-1-ylmethyl-5,6,7,8-tetrahydro-naphthalen-2-yloxy)-propyl]-amine

Mass spectrum (m/z) calcd for C₂₃H₃₈N₂O: 358.57; obsd. 359 (M+1).

### EXAMPLE 22

### (+)-1-{3-[3-(6-Pyrrolidin-1-ylmethyl-5,6,7,8-tetrahydro-naphthalen-2-yloxy)-propylamino]-propyl}-pyrrolidin-2-one; and

### (−)-1-{3-[3-(6-Pyrrolidin-1-ylmethyl-5,6,7,8-tetrahydro-naphthalen-2-yloxy)-propylamino]-propyl}-pyrrolidin-2-one

Mass spectrum (m/z) calcd for C₂₅H₃₉N₃O₂: 413.6; obsd. 414 (M+1).

### EXAMPLE 23

### (+)-N,N-Dimethyl-N′-[3-(6-pyrrolidin-1-ylmethyl-5,6,7,8-tetrahydro-naphthalen-2-yloxy)-propyl]-ethane-1,2-diamine

Mass spectrum (m/z) calcd for C₂₂H₃₇N₃O: 359.5; obsd. 360 (M+1).

### EXAMPLE 24

### (+)-(4-Chlorobenzyl)-[3-(6-pyrrolidin-1-ylmethyl-5,6,7,8-tetrahydro-naphthalen-2-yloxy)-propyl]-amine; and

### (−)-(4-Chlorobenzyl)-[3-(6-pyrrolidin-1-ylmethyl-5,6,7,8-tetrahydro-naphthalen-2-yloxy)-propyl]-amine

Mass spectrum (m/z) calcd for C₂₅H₃₃ClN₂O: 413.5; obsd. 414 (M+1), 416.

### EXAMPLE 25

### (−)-2-{Ethyl-[3-(6-pyrrolidin-1-ylmethyl-5,6,7,8-tetrahydro-naphthalen-2-yloxy)-propyl]-amino}-ethanol

Mass spectrum (m/z) calcd for C₂₂H₃₆N₂O₂: 360.54; obsd. 361 (M+1).

### EXAMPLE 26

### (+)-{1-[3-(6-Pyrrolidin-1-ylmethyl-5,6,7,8-tetrahydro-naphthalen-2-yloxy)-propyl]-piperidin-3-yl}-methanol

Mass spectrum (m/z) calcd for C₂₄H₃₈N₂O₂: 386.58; obsd. 387 (M+1).

### EXAMPLE 27

### (+)-(3,4-Dichlorobenzyl)-[3-(6-pyrrolidin-1-ylmethyl-5,6,7,8-tetrahydro-naphthalen-2-yloxy)-propyl]-amine; and

### (−)-(3,4-Dichlorobenzyl)-[3-(6-pyrrolidin-1-ylmethyl-5,6,7,8-tetrahydro-naphthalen-2-yloxy)-propyl]-amine

Mass spectrum (m/z) calcd for C₂₅H₃₂Cl₂N₂O: 447.45; obsd. 448 (M+1), 450, 452.

### EXAMPLE 28

### (+)-Benzyl-[3-(6-pyrrolidin-1-ylmethyl-5,6,7,8-tetrahydro-naphthalen-2-yloxy)-propyl]-amine; and

### (−)-Benzyl-[3-(6-pyrrolidin-1-ylmethyl-5,6,7,8-tetrahydro-naphthalen-2-yloxy)-propyl]-amine

Mass spectrum (m/z) calcd for C₂₅H₃₄N₂O: 378.56; obsd. 379 (M+1).

### EXAMPLE 29

### (+)-(2-Pyridin-2-ylethyl)-[3-(6-pyrrolidin-1-ylmethyl-5,6,7,8-tetrahydro-naphthalen-2-yloxy)-propyl]-amine; and

### (−)-(2-Pyridin-2-ylethyl)-[3-(6-pyrrolidin-1-ylmethyl-5,6,7,8-tetrahydro-naphthalen-2-yloxy)-propyl]-amine

Mass spectrum (m/z) calcd for C₂₅H₃₅N₃O: 393.5; obsd. 394 (M+1).

### EXAMPLE 30

### (+)-(4-tert-Butylcyclohexyl)-[3-(6-pyrrolidin-1-ylmethyl-5,6,7,8-tetrahydro-naphthalen-2-yloxy)-propyl]-amine; and

### (−)-(4-tert-Butylcyclohexyl)-[3-(6-pyrrolidin-1-ylmethyl-5,6,7,8-tetrahydro-naphthalen-2-yloxy)-propyl]-amine

Mass spectrum (m/z) calcd for C₂₈H₄₆N₂O: 426.68; obsd. 427 (M+1).

### EXAMPLE 31

### (+)-(3-Methoxybenzyl)-[3-(6-pyrrolidin-1-ylmethyl-5,6,7,8-tetrahydro-naphthalen-2-yloxy)-propyl]-amine; and

### (−)-(3-Methoxybenzyl)-[3-(6-pyrrolidin-1-ylmethyl-5,6,7,8-tetrahydro-naphthalen-2-yloxy)-propyl]-amine

Mass spectrum (m/z) calcd for C₂₆H₃₆N₂O₂: 408.58; obsd. 409 (M+1).

### EXAMPLE 32

### (+)-(4-Fluorobenzyl)-[3-(6-pyrrolidin-1-ylmethyl-5,6,7,8-tetrahydro-naphthalen-2-yloxy)-propyl]-amine; and

### (−)-(4-Fluorobenzyl)-[3-(6-pyrrolidin-1-ylmethyl-5,6,7,8-tetrahydro-naphthalen-2-yloxy)-propyl]-amine

Mass spectrum (m/z) calcd for C₂₅H₃₃FN₂O: 396.55; obsd. 397 (M+1).

### EXAMPLE 33

### (+)-(2,6-Difluorobenzyl)-[3-(6-pyrrolidin-1-ylmethyl-5,6,7,8-tetrahydro-naphthalen-2-yloxy)-propyl]-amine; and

### (−)-(2,6-Difluorobenzyl)-[3-(6-pyrrolidin-1-ylmethyl-5,6,7,8-tetrahydro-naphthalen-2-yloxy)-propyl]-amine

Mass spectrum (m/z) calcd for C₂₅H₃₂F₂N₂O: 414.54; obsd. 415 (M+1).

### EXAMPLE 34

### (+)-(3-Ethoxypropyl)-[3-(6-pyrrolidin-1-ylmethyl-5,6,7,8-tetrahydro-naphthalen-2-yloxy)-propyl]-amine; and

### (−)-(3-Ethoxypropyl)-[3-(6-pyrrolidin-1-ylmethyl-5,6,7,8-tetrahydro-naphthalen-2-yloxy)-propyl]-amine

Mass spectrum (m/z) calcd for C₂₃H₃₈N₂O₂: 374.57; obsd. 375 (M+1).

### EXAMPLE 35

### (+)-[3-(6-Pyrrolidin-1-ylmethyl-5,6,7,8-tetrahydro-naphthalen-2-yloxy)-propyl]-N-cyclohexyl-N-methylamine; and

### (−)-[3-(6-Pyrrolidin-1-ylmethyl-5,6,7,8-tetrahydro-naphthalen-2-yloxy)-propyl]-N-cyclohexyl-N-methylamine

Mass spectrum (m/z) calcd for C₂₅H₄₀N₂O: 384.6; obsd. 385 (M+1).

### EXAMPLE 36

### (+)-(2,2-Dimethylpropyl)-[3-(6-pyrrolidin-1-ylmethyl-5,6,7,8-tetrahydro-naphthalen-2-yloxy)-propyl]-amine; and

### (−)-(2,2-Dimethylpropyl)-[3-(6-pyrrolidin-1-ylmethyl-5,6,7,8-tetrahydro-naphthalen-2-yloxy)-propyl]-amine

Mass spectrum (m/z) calcd for C₂₃H₃₈N₂O: 358.57; obsd. 359 (M+1).

### EXAMPLE 37

### (+)-1-Cyclohexyl-4-[3-(6-pyrrolidin-1-ylmethyl-5,6,7,8-tetrahydro-naphthalen-2-yloxy)-propyl]-piperazine; and

### (−)-1-Cyclohexyl-4-[3-(6-pyrrolidin-1-ylmethyl-5,6,7,8-tetrahydro-naphthalen-2-yloxy)-propyl]-piperazine

Mass spectrum (m/z) calcd for C₂₈H₄₅N₃O: 439.68; obsd. 4440 (M+1).

### EXAMPLE 38

### (+)-1-[3-(6-Pyrrolidin-1-ylmethyl-5,6,7,8-tetrahydro-naphthalen-2-yloxy)-propyl]-piperidine-4-carboxylic acid amide; and

### (−)-1-[3-(6-Pyrrolidin-1-ylmethyl-5,6,7,8-tetrahydro-naphthalen-2-yloxy)-propyl]-piperidine-4-carboxylic acid amide

Mass spectrum (m/z) calcd for C₂₄H₃₇N₃O₂: 399.57; obsd. 400 (M+1).

### EXAMPLE 39

### (+)-1-{4-[3-(6-Pyrrolidin-1-ylmethyl-5,6,7,8-tetrahydro-naphthalen-2-yloxy)-propyl]-piperazin-1-yl}-ethanone; and

### (−)-1-{4-[3-(6-Pyrrolidin-1-ylmethyl-5,6,7,8-tetrahydro-naphthalen-2-yloxy)-propyl]-piperazin-1-yl}-ethanone

Mass spectrum (m/z) calcd for C₂₄H₃₇N₃O₂: 399.57; obsd. 400 (M+1).

### EXAMPLE 40

### (+)-3-Methyl-1-[3-(6-pyrrolidin-1-ylmethyl-5,6,7,8-tetrahydro-naphthalen-2-yloxy)-propyl]-piperidine; and

### (−)-3-Methyl-1-[3-(6-pyrrolidin-1-ylmethyl-5,6,7,8-tetrahydro-naphthalen-2-yloxy)-propyl]-piperidine

Mass spectrum (m/z) calcd for C₂₄H₃₈N₂O: 370.59; obsd. 371 (M+1).

### EXAMPLE 41

### (+)-2-Methyl-1-[3-(6-pyrrolidin-1-ylmethyl-5,6,7,8-tetrahydro-naphthalen-2-yloxy)-propyl]-piperidine; and

### (−)-2-Methyl-1-[3-(6-pyrrolidin-1-ylmethyl-5,6,7,8-tetrahydro-naphthalen-2-yloxy)-propyl]-piperidine

Mass spectrum (m/z) calcd for C₂₄H₃₈N₂O: 370.58; obsd. 371+1).

### EXAMPLE 42

### (+)-4-Methyl-1-[3-(6-pyrrolidin-1-ylmethyl-5,6,7,8-tetrahydro-naphthalen-2-yloxy)-propyl]-piperidine; and

### (−)-4-Methyl-1-[3-(6-pyrrolidin-1-ylmethyl-5,6,7,8-tetrahydro-naphthalen-2-yloxy)-propyl]-piperidine

Mass spectrum (m/z) calcd for C₂₄H₃₈N₂O: 370.58; obsd. 371+1).

### EXAMPLE 43

### (+)-{1-[3-(6-Pyrrolidin-1-ylmethyl-5,6,7,8-tetrahydro-naphthalen-2-yloxy)-propyl]-pyrrolidin-3-yl}-diethylamine; and

### (−)-{1-[3-(6-Pyrrolidin-1-ylmethyl-5,6,7,8-tetrahydro-naphthalen-2-yloxy)-propyl]-pyrrolidin-3-yl}-diethylamine

Mass spectrum (m/z) calcd for C₂₆H₄₃N₃O: 413.65; obsd. 414 (M+1).

### EXAMPLE 44

### (+)-Methyl-(1-methylpiperidin-4-yl)-[3-(6-pyrrolidin-1-ylmethyl-5,6,7,8-tetrahydro-naphthalen-2-yloxy)-propyl]-amine; and

### (−)-Methyl-(1-methylpiperidin-4-yl)-[3-(6-pyrrolidin-1-ylmethyl-5,6,7,8-tetrahydro-naphthalen-2-yloxy)-propyl]-amine

Mass spectrum (m/z) calcd for C₂₅H₄₁N₃O: 399.62; obsd. 400 (M+1).

### EXAMPLE 45

### (+)-1-(3,5-Dichloropyridin-4-yl)-4-[3-(6-pyrrolidin-1-ylmethyl-5,6,7,8-tetrahydro-naphthalen-2-yloxy)-propyl]-piperazine; and

### (−)-1-(3,5-Dichloropyridin-4-yl)-4-[3-(6-pyrrolidin-1-ylmethyl-5,6,7,8-tetrahydro-naphthalen-2-yloxy)-propyl]-piperazine

Mass spectrum (m/z) calcd for C₂₇H₃₆Cl₂N₄O: 503.51; obsd. 504 (M+1), 506, 508.

### EXAMPLE 46

### (+)-2-{4-[3-(6-Pyrrolidin-1-ylmethyl-5,6,7,8-tetrahydro-naphthalen-2-yloxy)-propyl]-piperazin-1-yl}-pyrimidine; and

### (−)-2-{4-[3-(6-Pyrrolidin-1-ylmethyl-5,6,7,8-tetrahydro-naphthalen-2-yloxy)-propyl]-piperazin-1-yl}-pyrimidine

Mass spectrum (m/z) calcd for C₂₆H₃₇N₅O: 435.61; obsd. 436 (M+1).

### EXAMPLE 47

### (+)-2,2,N,N-Tetramethyl-N′-[3-(6-pyrrolidin-1-ylmethyl-5,6,7,8-tetrahydro-naphthalen-2-yloxy)-propyl]-propane-1,3-diamine; and

### (−)-2,2,N,N-Tetramethyl-N′-[3-(6-pyrrolidin-1-ylmethyl-5,6,7,8-tetrahydro-naphthalen-2-yloxy)-propyl]-propane-1,3-diamine

Mass spectrum (m/z) calcd for C₂₅H₄₃N₃O: 401.63; obsd. 402 (M+1).

### EXAMPLE 48

### (+)-2-[3-(6-Pyrrolidin-1-ylmethyl-5,6,7,8-tetrahydro-naphthalen-2-yloxy)-propyl]-2,3-dihydro-1H-isoindole; and

### (−)-2-[3-(6-Pyrrolidin-1-ylmethyl-5,6,7,8-tetrahydro-naphthalen-2-yloxy)-propyl]-2,3-dihydro-1H-isoindole

Mass spectrum (m/z) calcd for C₂₆H₃₄N₂O: 390.57; obsd. 391 (M+1).

### EXAMPLE 49

### (+)-{1-[3-(6-Pyrrolidin-1-ylmethyl-5,6,7,8-tetrahydro-naphthalen-2-yloxy)-propylamino]-cyclopentyl}-methanol; and

### (−)-{1-[3-(6-Pyrrolidin-1-ylmethyl-5,6,7,8-tetrahydro-naphthalen-2-yloxy)-propylamino]-cyclopentyl}-methanol

Mass spectrum (m/z) calcd for C₂₄H₃₈N₂O₂: 386.58; obsd. 387 (M+1).

### EXAMPLE 50

### (+)-2,6-Dimethyl-4-[3-(6-pyrrolidin-1-ylmethyl-5,6,7,8-tetrahydro-naphthalen-2-yloxy)-propyl]-morpholine; and

### (−)-2,6-Dimethyl-4-[3-(6-pyrrolidin-1-ylmethyl-5,6,7,8-tetrahydro-naphthalen-2-yloxy)-propyl]-morpholine

Mass spectrum (m/z) calcd for C₂₄H₃₈N₂O₂: 386.68; obsd. 387 (M+1).

### EXAMPLE 51

### (+)-(1-Methyl-2-morpholin-4-ylmethyl)-[3-(6-pyrrolidin-1-ylmethyl-5,6,7,8-tetrahydro-naphthalen-2-yloxy)-propyl]-amine; and

### (−)-(1-Methyl-2-morpholin-4-ylethyl)-[3-(6-pyrrolidin-1-ylmethyl-5,6,7,8-tetrahydro-naphthalen-2-yloxy)-propyl]-amine

Mass spectrum (m/z) calcd for C₂₅H₄₁N₃O₂: 415.62; obsd. 416 (M+1).

### EXAMPLE 52

### (+)-Cyclohexylmethyl-[3-(6-pyrrolidin-1-ylmethyl-5,6,7,8-tetrahydro-naphthalen-2-yloxy)-propyl]-amine; and

### (−)-Cyclohexylmethyl-[3-(6-pyrrolidin-1-ylmethyl-5,6,7,8-tetrahydro-naphthalen-2-yloxy)-propyl]-amine

Mass spectrum (m/z) calcd for C₂₅H₄₀N₂O: 384.6; obsd. 385 (M+1).

### EXAMPLE 53

### (+)-(4-Morpholin-4-yl-tetrahydrofuran-3-yl)-[3-(6-pyrrolidin-1-ylmethyl-5,6,7,8-tetrahydro-naphthalen-2-yloxy)-propyl]-amine; and

### (−)-(4-Morpholin-4-yl-tetrahydrofuran-3-yl)-[3-(6-pyrrolidin-1-ylmethyl-5,6,7,8-tetrahydro-naphthalen-2-yloxy)-propyl]-amine

Mass spectrum (m/z) calcd for C₂₆H₄₁N₃O₃: 443.63; obsd. 444 (M+1).

### EXAMPLE 54

### (+)-(1-Methyl-1-phenylethyl)-[3-(6-pyrrolidin-1-ylmethyl-5,6,7,8-tetrahydro-naphthalen-2-yloxy)-propyl]-amine; and

### (−)-(1-Methyl-1-phenylethyl)-[3-(6-pyrrolidin-1-ylmethyl-5,6,7,8-tetrahydro-naphthalen-2-yloxy)-propyl]-amine

Mass spectrum (m/z) calcd for C₂₇H₃₈N₂O: 406.61; obsd. 407 (M+1).

### EXAMPLE 55

### (+)-Furan-2-ylmethyl-[3-(6-pyrrolidin-1-ylmethyl-5,6,7,8-tetrahydro-naphthalen-2-yloxy)-propyl]-methylamine; and

### (−)-Furan-2-ylmethyl-[3-(6-pyrrolidin-1-ylmethyl-5,6,7,8-tetrahydro-naphthalen-2-yloxy)-propyl]-methylamine

Mass spectrum (m/z) calcd for C₂₄H₃₄N₂O₂: 382.55; obsd. 383 (M+1).

### EXAMPLE 56

### (+)-Furan-2-ylmethyl-[3-(6-pyrrolidin-1-ylmethyl-5,6,7,8-tetrahydro-naphthalen-2-yloxy)-propyl]-amine; and

### (−)-Furan-2-ylmethyl-[3-(6-pyrrolidin-1-ylmethyl-5,6,7,8-tetrahydro-naphthalen-2-yloxy)-propyl]-amine

Mass spectrum (m/z) calcd for C₂₃H₃₂N₂O₂: 368.52; obsd. 369 (M+1).

### EXAMPLE 57

### (+)-2-{1-[3-(6-Pyrrolidin-1-ylmethyl-5,6,7,8-tetrahydro-naphthalen-2-yloxy)-propyl]-pyrrolidin-3-yl}-pyridine; and

### (−)-2-{1-[3-(6-Pyrrolidin-1-ylmethyl-5,6,7,8-tetrahydro-naphthalen-2-yloxy)-propyl]-pyrrolidin-3-yl}-pyridine

Mass spectrum (m/z) calcd for C₂₇H₃₇N₃O: 419.61; obsd. 420 (M+1).

### EXAMPLE 58

### (+)-[3-(6-Pyrrolidin-1-ylmethyl-5,6,7,8-tetrahydro-naphthalen-2-yloxy)-propyl]-(tetrahydro-pyran-4-ylmethyl)-amine; and

### (−)-[3-(6-Pyrrolidin-1-ylmethyl-5,6,7,8-tetrahydro-naphthalen-2-yloxy)-propyl]-(tetrahydro-pyran-4-ylmethyl)-amine

Mass spectrum (m/z) calcd for C₂₄H₃₈N₂O₂: 386.58; obsd. 387 (M+1).

### EXAMPLE 59

### (+)-2-{1-[3-(6-Pyrrolidin-1-ylmethyl-5,6,7,8-tetrahydro-naphthalen-2-yloxy)-propyl]-pyrrolidin-2-yl}-pyridine; and

### (−)-2-{1-[3-(6-Pyrrolidin-1-ylmethyl-5,6,7,8-tetrahydro-naphthalen-2-yloxy)-propyl]-pyrrolidin-2-yl}-pyridine

Mass spectrum (m/z) calcd for C₂₇H₃₇N₃O: 419.61; obsd. 420 (M+1).

### EXAMPLE 60

### (+)-(1-isopropyl-piperidin-4-ylmethyl)-[3-(6-pyrrolidin-1-ylmethyl-5,6,7,8-tetrahydro-naphthalen-2-yloxy)-propyl]-amine; and

### (−)-(1-isopropyl-piperidin-4-ylmethyl)-[3-(6-pyrrolidin-1-ylmethyl-5,6,7,8-tetrahydro-naphthalen-2-yloxy)-propyl]-amine

Mass spectrum (m/z) calcd for C₂₇H₄₅N₃O: 427.67; obsd. 428 (M+1).

### EXAMPLE 61

### (+)-[2-(4-Fluorophenyl)-ethyl]-[3-(6-pyrrolidin in-1-ylmethyl-5,6,7,8-tetrahydro-naphthalen-2-yloxy)-propyl]-amine; and

### (−)-[2-(4-Fluorophenyl)-ethyl]-[3-(6-pyrrolidin-1-ylmethyl-5,6,7,8-tetrahydro-naphthalen-2-yloxy)-propyl]-amine

Mass spectrum (m/z) calcd for C₂₆H₃₅FN₂O: 410.57; obsd. 411 (M+1).

### EXAMPLE 62

### (+)-2-Methyl-6-[3-(6-pyrrolidin-1-ylmethyl-5,6,7,8-tetrahydro-naphthalen-2-yloxy)-propylamino]-heptan-2-ol; and

### (−)-2-Methyl-6-[3-(6-pyrrolidin-1-ylmethyl-5,6,7,8-tetrahydro-naphthalen-2-yloxy)-propylamino]-heptan-2-ol

Mass spectrum (m/z) calcd for C₂₆H₄₄N₂O₂: 416.65; obsd. 417 (M+1).

### EXAMPLE 63

### (+)-(1-isopropyl-piperidin-3-ylmethyl)-[3-(6-pyrrolidin-1-ylmethyl-5,6,7,8-tetrahydro-naphthalen-2-yloxy)-propyl]-amine; and

### (−)-(1-Isopropyl-piperidin-3-ylmethyl)-[3-(6-pyrrolidin-1-ylmethyl-5,6,7,8-tetrahydro-naphthalen-2-yloxy)-propyl]-amine

Mass spectrum (m/z) calcd for C₂₇H₄₅N₃O: 427.67; obsd. 428 (M+1).

### EXAMPLE 64

### (+)-[3-(6-pyrrolidin-1-ylmethyl-5,6,7,8-tetrahydro-naphthalen-2-yloxy)-propyl]-cyclohexyl-amine; and

### (−)-[3-(6-pyrrolidin-1-ylmethyl-5,6,7,8-tetrahydro-naphthalen-2-yloxy)-propyl]-cyclohexyl-amine

Mass spectrum (m/z) calcd for C₂₄H₃₈N₂O: 370.58; obsd. 371 (M+1).

### EXAMPLE 65

### (+)-6-[3-(6-Pyrrolidin-1-ylmethyl-5,6,7,8-tetrahydro-naphthalen-2-yloxy)-propyl]-1-6-azabicyclo-[3.2.1]octane; and

### (−)-6-[3-(6-Pyrrolidin-1-ylmethyl-5,6,7,8-tetrahydro-naphthalen-2-yloxy)-propyl]-6-azabicyclo-[3.2.1]octane

Mass spectrum (m/z) calcd for C₂₅H₃₈N₂O: 382.59; obsd. 383 (M+1).

### EXAMPLE 66

### (+)-4-Methoxy-1-[3-(6-pyrrolidin-1-ylmethyl-5,6,7,8-tetrahydro-naphthalen-2-yloxy)-propyl]-piperidine; and

### (−)-4-Methoxy-1-[3-(6-pyrrolidin-1-ylmethyl-5,6,7,8-tetrahydro-naphthalen-2-yloxy)-propyl]-piperidine

Mass spectrum (m/z) calcd for C₂₄H₃₈N₂O₂: 386.58; obsd. 387 (M+1).

### EXAMPLE 67

### (+)-(2-{1-[3-(6-pyrrolidin-1-ylmethyl-5,6,7,8-tetrahydro-naphthalen-2-yloxy)-propyl]-piperidin-4-yl}-ethyl)-dimethylamine; and

### (−)-(2-{1-[3-(6-pyrrolidin-1-ylmethyl-5,6,7,8-tetrahydro-naphthalen-2-yloxy)-propyl]-piperidin-4-yl}-ethyl)-dimethylamine

Mass spectrum (m/z) calcd for C₂₇H₄₅N₃O: 427.67; obsd. 428 (M+1).

### EXAMPLE 68

### (+)-2,6-Dimethyl-4-{1-[3-(6-pyrrolidin-1-ylmethyl-5,6,7,8-tetrahydro-naphthalen-2-yloxy)-propyl]-piperidin-4-yl-}-morpholine; and

### (−)-2,6-Dimethyl-4-{1-[3-(6-pyrrolidin-1-ylmethyl-5,6,7,8-tetrahydro-naphthalen-2-yloxy)-propyl]-piperidin-4-yl}-morpholine

Mass spectrum (m/z) calcd for C₂₉H₄₇N₃O: 469.71; obsd. 470 (M+1).

### EXAMPLE 69

### (+)-2-[3-(6-Pyrrolidin-1-ylmethyl-5,6,7,8-tetrahydro-naphthalen-2-yloxy)-propyl]-octahydro-pyrrolo[1,2-a]pyrazine; and

### (−)-2-[3-(6-Pyrrolidin-1-ylmethyl-5,6,7,8-tetrahydro-naphthalen-2-yloxy)-propyl]-octahydro-pyrrolo[1,2-a]pyrazine

Mass spectrum (m/z) calcd for C₂₅H₃₉N₃O: 397.6; obsd. 398 (M+1).

### EXAMPLE 70

### (+)-(1H-Imidazol-2-ylmethyl)-methyl-[3-(6-pyrrolidin-1-ylmethyl-5,6,7,8-tetrahydro-naphthalen-2-yloxy)-propyl]-amine; and

### (−)-(1H-Imidazol-2-ylmethyl)-methyl-[3-(6-Pyrrolidin-1-ylmethyl-5,6,7,8-tetrahydro-naphthalen-2-yloxy)-propyl]-amine

Mass spectrum (m/z) calcd for C₂₃H₃₄N₄O: 382.55; obsd. 383 (M+1).

### EXAMPLE 71

### (+)-2-(4-Fluorophenyl)-1-[3-(6-pyrrolidin-1-ylmethyl-5,6,7-8-tetrahydro-naphthalen-2-yloxy)-propyl]-pyrrolidine; and

### (−)-2-(4-Fluorophenyl)-1-[3-(6-pyrrolidin in-i -ylmethyl-5,6,7-8-tetrahydro-naphthalen-2-yloxy)-propyl]-pyrrolidine

Mass spectrum (m/z) calcd for C₂₈H₃₇FN₂O: 436.61; obsd. 437 (M+1).

### EXAMPLE 72

### (+)-(2-{1-[3-(6-pyrrolidin-1-ylmethyl-5,6,7,8-tetrahydro-naphthalen-2-yloxy)-propyl]-piperidin-4-yloxy}-ethyl)-dimethylamine; and

### (−)-(2-{1-[3-(6-pyrrolidin-1-ylmethyl-5,6,7,8-tetrahydro-naphthalen-2-yloxy)-propyl]-piperidin-4-yloxy}-ethyl)-dimethylamine

Mass spectrum (m/z) calcd for C₂₇H₄₅N₃O₂: 443.67; obsd. 444 (M+1).

### EXAMPLE 73

### (−)-(2-Pyrrolidin-1-ylethyl)-[3-(6-pyrrolidin-1-ylmethyl-5,6,7,8-tetrahydro-naphthalen-2-yloxy)-propyl]-amine

Mass spectrum (m/z) calcd for C₂₄H₃₉N₃O: 385.6; obsd. 386 (M+1).

### EXAMPLE 74

### (−)-(2-Chlorobenzyl)-[3-(6-pyrrolidin-1-ylmethyl-5,6,7,8-tetrahydro-naphthalen-2-yloxy)-propyl]-amine

Mass spectrum (m/z) calcd for C₂₅H₃₃ClN₂O: 413.6; obsd. 414 (M+1).

### EXAMPLE 75

### (−)-Pyridin-3-ylmethyl-[3-(6-pyrrolidin-1-ylmethyl-5,6,7,8-tetrahydro-naphthalen-2-yloxy)-propyl]-amine

Mass spectrum (m/z) calcd for C₂₄H₃₃N₃O: 379.6; obsd. 380 (M+1).

### EXAMPLE 76

### (−)-(2-Piperidin-1-ylethyl)-[3-(6-pyrrolidin-1-ylmethyl-5,6,7,8-tetrahydro-naphthalen-2-yloxy)-propyl]-amine

Mass spectrum (m/z) calcd for C₂₅H₄₁N₃O: 399.6; obsd. 400 (M+1).

### EXAMPLE 77

### (−)-(3,4-Difluorobenzyl)-[3-(6-pyrrolidin-1-ylmethyl-5,6,7,8-tetrahydro-naphthalen-2-yloxy)-propyl]-amine

Mass spectrum (m/z) calcd for C₂₅H₃₂F₂N₂O: 414.5; obsd. 415 (M+1).

### EXAMPLE 78

### (−)-N,N,N′-Trimethyl-N′-[3-(6-pyrrolidin-1-ylmethyl-5,6,7,8-tetrahydro-naphthalen-2-yloxy)-propyl]-ethane-1,2-diamine

Mass spectrum (m/z) calcd for C₂₃H₃₉N₃O: 373.6 obsd. 374 (M+1).

### EXAMPLE 79

### (−)-(2,4-Dichlorobenzyl)-[3-(6-pyrrolidin-1-ylmethyl-5,6,7,8-tetrahydro-naphthalen-2-yloxy)-propyl]-amine

Mass spectrum (m/z) calcd for C₂₅H₃₂Cl₂N₂O: 447.5; obsd. 448 (M+1).

### EXAMPLE 80

### (−)-(3-Imidazol-1-yl-propyl)-[3-(6-pyrrolidin-1-ylmethyl-5,6,7,8-tetrahydro-naphthalen-2-yloxy)-propyl]-amine

Mass spectrum (m/z) calcd for C₂₄H₃₆N₄O: 396.6; obsd. 397 (M+1).

### EXAMPLE 81

### (−)-3,5-Dimethyl-1-[3-(6-pyrrolidin-1-ylmethyl-5,6,7,8-tetrahydro-naphthalen-2-yloxy)-propyl]-piperidine

Mass spectrum (m/z) calcd for C₂₅H₄₀N₂O: 384.6; obsd. 385 (M+1).

### EXAMPLE 82

### (−)-1-Pyridin-2-yl-4-[3-(6-pyrrolidin-1-ylmethyl-5,6,7,8-tetrahydro-naphthalen-2-yloxy)-propyl]-piperazine

Mass spectrum (m/z) calcd for C₂₇H₃₈N₄O: 434.6; obsd. 435 (M+1).

### EXAMPLE 83

### (−)-(3-Azepan-1-ylpropyl)-[3-(6-pyrrolidin-1-ylmethyl-5,6,7,8-tetrahydro-naphthalen-2-yloxy)-propyl]-amine

Mass spectrum (m/z) calcd for C₂₇H₄₅N₃O: 427.7; obsd. 428 (M+1).

### EXAMPLE 84

### (−)-1-[3-(6-Pyrrolidin-1-ylmethyl-5,6,7,8-tetrahydro-naphthalen-2-yloxy)-propyl]-1,2,3,4,5,6-hexahydro-[4,4′]bipyridinyl

Mass spectrum (m/z) calcd for C₂₈H₃₉N₃O: 433.6; obsd. 434 (M+1).

### EXAMPLE 85

### (−)-Furan-3-ylmethyl-[3-(6-pyrrolidin-1-ylmethyl-5,6,7,8-tetrahydro-naphthalen-2-yloxy)-propyl]-amine

Mass spectrum (m/z) calcd for C₂₃H₃₂N₂O₂: 368.5; obsd. 369 (M+1).

### EXAMPLE 86

### (−)-1,6-Dimethyl-2-[3-(6-pyrrolidin-1-ylmethyl-5,6,7,8-tetrahydro-naphthalen-2-yloxy)-propyl]-1,2,3,4-tetrahydro-pyrrolo[1,2-a]pyrazine

Mass spectrum (m/z) calcd for C₂₇H₃₉N₃O: 421.6; obsd. 422 (M+1).

### EXAMPLE 87

### (−)-[3-(6-Pyrrolidin-1-ylmethyl-5,6,7,8-tetrahydro-naphthalen-2-yloxy)-propyl]-[2-(tetrahydro-pyran-2-yl)-ethyl]-amine

Mass spectrum (m/z) calcd for C₂₅H₄₀N₂O₂: 400.60; obsd. 401 (M+1).

### EXAMPLE 88

### (−)-(2-Imidazol-1-yl-ethyl)-[3-(6-pyrrolidin-1-ylmethyl-5,6,7,8-tetrahydro-naphthalen-2-yloxy)-propyl]-amine

Mass spectrum (m/z) calcd for C₂₃H₃₄N₄O₂: 382.6; obsd. 383 (M+1).

### EXAMPLE 89

### (−)-(1H-Imidazol-2-ylmethyl)-[3-(6-pyrrolidin-1-ylmethyl-5,6,7,8-tetrahydro-naphthalen-2-yloxy)-propyl]-amine

Mass spectrum (m/z) calcd for C₂₂H₃₂N₄O: 368.5; obsd. 369 (M+1).

### EXAMPLE 90

### (−)-1-Cyclopentyl-4-[3-(6-pyrrolidin-1-ylmethyl-5,6,7,8-tetrahydro-naphthalen-2-yloxy)-propyl]-piperazin-2-one

Mass spectrum (m/z) calcd for C₂₇H₄₁N₃O₂: 439.6; obsd. 440 (M+1).

### EXAMPLE 91

### (−)-[3-(6-Pyrrolidin-1-ylmethyl-5,6,7,8-tetrahydro-naphthalen-2-yloxy)-propyl]-(tetrahydro-pyran-4-ylmethyl)-methylamine

Mass spectrum (m/z) calcd for C₂₅H₄₀N₂O₂: 440.60; obsd. 441 (M+1).

### EXAMPLE 92

### (−)-1-[2-(6-Pyrrolidin-1-ylmethyl-5,6,7,8-tetrahydro-naphthalen-2-yloxy)-ethyl]-piperidine dihydrochloride

M.P. 265.5-266.8° C.

Mass spectrum (m/z) calcd for C₂₂H₃₄N₂O: 342.50; obsd. 343 (M+1).

### Determination of Biological Activity

The in vitro affinity of the compounds in the present invention at the rat or human histamine H3 receptors can be determined according to the following procedure. Frozen rat frontal brain or frozen human post-mortem frontal brain is homogenized in 20 volumes of cold 50 mM Tris.HCl containing 2 mM MgCl₂ (pH to 7.4 at 4° C.). The homogenate is then centrifuged at 45,000 G for 10 minutes. The supernatant is decanted and the membrane pellet re-suspended by Polytron in cold 50 mM Tris.HCl containing 2 mM MgCl₂ (pH to 7.4 at 4 degrees C.) and centrifuged again. The final pellet is re-suspended in 50 mM Tris HCl containing 2 mM MgCl₂ (pH to 7.4 at 25 degrees C.) at a concentration of 12 mg/mL. Dilutions of compounds are made in 10% DMSO/50 mM Tris buffer (pH 7.4) (at 10×final concentration, so that the final DMSO concentration is 1%). Incubations are initiated by the addition of membranes (200 microliters) to 96-well V-bottom polypropylene plates containing 25 microliters of drug dilutions and 25 microliters of radioligand (1 nM final concentration ³H-N-methylhistamine). After a 1-hour incubation, assay samples are rapidly filtered through Whatman GF/B filters and rinsed with ice-cold 50 mM Tris buffer (pH 7.4) using a Skatron cell harvester. Radioactivity is quantified using a BetaPlate scintillation counter. The percent inhibition of specific binding can then be determined for each dose of the compound, and an IC50 or Ki value can be calculated from these results.

TABLE 1
Rat H3 Binding for selected compounds

| | Rat H3 activity |
|---|---|
| Example # | (Kᵢ, nM) |
| 3 (±) | 8.4 |
| 6 (±) | 9.0 |
| 7 (±) | 3.0 |
| 10 (−) | 1.1 |
| 15 (−) | 5.0 |
| 16 (−) | 7.2 |
| 24 (−) | 0.84 |
| 27 (−) | 1.58 |
| 28 (−) | 4.7 |
| 32 (−) | 1.25 |
| 40 (+) | 8.4 |
| 40 (−) | 3.54 |
| 41 (−) | 5.89 |
| 42 (−) | 4.32 |
| 51 (−) | 6.2 |
| 55 (−) | 4.5 |
| 64 (−) | 4.36 |
| 65 (−) | 2.11 |
| 71 (+) | 9.6 |
| 77 (−) | 1.95 |
| 79 (−) | 1.9 |
| 81 (−) | 9.14 |
| 92 (−) | 11.3 |

1. A compound of formula I 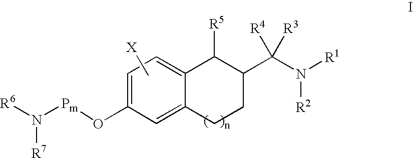
or the pharmaceutically acceptable salt(s) thereof, wherein:
m=2, 3 or 4;
n=1 or 2;
P is independently selected from the group consisting of CH₂, C₂-C₆ alkyl or cycloalkyl group optionally substituted by hydrogen, C₁-C₆ alkyl or OH;
R¹ and R² together with the nitrogen to which they are attached form a 3-10 member cyclic or bicyclic ring, optionally substituted with up to two additional heteroatoms selected from the group consisting of N, O or S [including the cyclic or bicyclic rings azetidine, pyrrolidine, piperidine, azepine piperazine, morpholine and thiomorpholine] and optionally substituted with H, C₁-C₆ alkyl or cycloalkyl groups, aryl or heteroaryl rings, or oxygen (including sulfoxide or sulfone); R¹ and R² are independently selected from the group that includes hydrogen, C₁-C₆ alkyl or C₃-C₇cycloalkyl;
R³, R⁴ and R⁵ are independently selected from the group consisting of hydrogen, and C₁-C₆ alkyl;
R⁶ and R⁷ are independently selected from the group consisting of hydrogen, C₁-C₁₂ alkyl, C₃-C₈ cycloalkyl, aryl or heteroaryl optionally substituted with up to three of Y; or
R⁶ and R⁷ together with the nitrogen to which they are attached form a 3-10 member cyclic or bicyclic ring optionally substituted with up to two additional heteroatoms selected from N, O or S [including the cyclic or bicyclic rings azetidine, pyrrolidine, piperidine, azepine, piperazine, morpholine and thiomorpholine] and optionally substituted by up to three groups of R²¹, O(═C)—R²¹ and CONR²²R²³
wherein R²¹, R²² and R²³ are independently selected from the group consisting of H, C₁-C₆ alkyl, aryl, C₃-C₈ cycloalkyl, hydroxyl, hydroxyalkyl, alkoxyalkyl, arylalkyl, heteroarylalkyl; and
X and Y are independently selected from the group consisting of H, F, Cl, Br, I, CN, OH, NR¹⁵R¹⁶, CF₃, C₂F₅, C₁-C₆ alkyl or C₃-C₈ cycloalkyl, aryl and heteroaryl [optionally substituted with halogen, C₁-C₆ alkyl, C₁-C₆ alkoxy, CF₃], C₁-C₆ alkoxy, and C₁-C₆alkyl-S(O)_{q}—, wherein q is 0, 1, 2 and R¹⁵ and R¹⁶ are independently selected from R¹ and R².

2. The compound of claim 1, wherein R¹ and R² together with the nitrogen to which they are attached form a piperidine ring.

3. The compound of claim 1, wherein R¹ and R² together with the nitrogen to which they are attached form a pyrrolidine ring.

4. The compound of claim 2, wherein m is three, n is one and R³ and R⁴ are either hydrogen or methyl.

5. The compound of claim 3, wherein m is three, n is one and R³ and R⁴ are either hydrogen or methyl.

6. The compound of claim 1, wherein R¹ and R² together with the nitrogen to which they are attached form a piperidine ring, m is three, n is one and k is 0 and R³, R⁴ and R⁵ are hydrogen or methyl.

7. The compound of claim 6, wherein R³, R⁴ and R⁵ are hydrogen and R⁶ is phenyl.

8. The compound (+)-6-pyrrolidin-1-ylmethyl-5,6,7,8-tetrahydro-naphthalen-2 ol.

9. The compound (−)-1-[6-(3-chloropropoxy)-1,2,3,4-tetrahydro-naphthalen-2 ylmethyl]-pyrrolidine.

10. The compounds of formula I wherein the compound is selected from the group consisting of:
4-[6-(2-piperidin-1-ylethoxy)-1,2,3,4-tetrahydro-naphthalen-2-ylmethyl]-morpholine;
1-methyl-4-[6-(2-piperidin-1-ylethoxy)-1,2,3,4-tetrahydro-naphthalen-2-ylmethyl]-piperazine;
1-methyl-4-{1-[6-(2-piperidin-1-ylethoxy)-1,2,3,4-tetrahydro-naphthalen-2-yl]-ethyl}-piperazine;
tert-butyl-{1-[6-(2-piperidin-1-ylethoxy)-1,2,3,4-tetrahydro-naphthalen-2-yl]-ethyl}-methylamine;
1-[2-(5-methyl-6-pyrrolidin-1-ylmethyl-5,6,7,8-tetrahydro-naphthalen-2-yloxy)-ethyl]-piperidine;
1-[2-(6-pyrrolidin-1-ylmethyl-5,6,7,8-tetrahydro-naphthalen-2-yloxy)-ethyl]-pyrrolidine;
1-[2-(6-(2,5-dimethyl)pyrrolidin-1-ylmethyl-5,6,7,8-tetrahydro-naphthalen-2-yloxy)-ethyl]-pyrrolidine;
N-benzyl-[2-(6-pyrrolidin-1-ylmethyl-5,6,7,8-tetrahydro-naphthalen-2-yloxy)-ethyl]-methylamine;
1-[2-(3-methyl-6-pyrrolidin-1-ylmethyl-5,6,7,8-tetrahydro-naphthalen-2-yloxy)-ethyl]-piperidine;
1-[2-(1,3-difluoro-6-pyrrolidin-1-ylmethyl-5,6,7,8-tetrahydro-naphthalen-2-yloxy)-ethyl]-piperidine;
1-[2-(1-fluoro-6-pyrrolidin-1-ylmethyl-5,6,7,8-tetrahydro-naphthalen-2-yloxy)-ethyl]-piperidine;
1-[2-(2-pyrrolidin-1-ylmethylindan-5-yloxy)-ethyl]-piperidine;
1-[3-(2-pyrrolidin-1-ylmethyl-indan-5-yloxy)-propyl]-pyrrolidine;
2-[3-(6-pyrrolidin-1-ylmethyl-5,6,7,8-tetrahydro-naphthalen-2-yloxy)-propyl]-octahydro-isoindole;
2-[3-(6-pyrrolidin-1-ylmethyl-5,6,7,8-tetrahydro-naphthalen-2-yloxy)-propyl]-octahydro-cyclopenta[c]pyrrole;
2-methyl-5-[3-(6-pyrrolidin-1-ylmethyl-5,6,7,8-tetrahydro-naphthalen-2-yloxy)-propyl]-octahydro-pyrrolo[3,4-c]pyrrole;
pyridin-4-yl-[3-(6-pyrrolidin-1-ylmethyl-5,6,7,8-tetrahydro-naphthalen-2-yloxy)-propyl]-methylamine;
pyrimidin-2-yl-[3-(6-pyrrolidin-1-ylmethyl-5,6,7,8-tetrahydro-naphthalen-2-yloxy)-propyl]-methylamine;
isopropyl-[3-(6-pyrrolidin-1-ylmethyl-5,6,7,8-tetrahydro-naphthalen-2-yloxy)-propyl]-phenylamine;
cyclopentyl-[3-(6-pyrrolidin-1-ylmethyl-5,6,7,8-tetrahydro-naphthalen-2-yloxy)-propyl]-isopropylamine;
2,5-dimethyl-1-[3-(6-pyrrolidin-1-ylmethyl-5,6,7,8-tetrahydro-naphthalen-2-yloxy)-propyl]-pyrrolidine;
1-[4-(6-pyrrolidin-1-ylmethyl-5,6,7,8-tetrahydro-naphthalen-2-yloxy)-butyl]-pyrrolidine;
1-[3-(6-pyrrolidin-1-ylmethyl-6,7,8,9-tetrahydro-5H-benzocyclohepten-2-yloxy)-propyl]-piperidine;
1-[2-(6-pyrrolidin-1-ylmethyl-6,7,8,9-tetrahydro-5H-benzocyclohepten-2-yloxy)-ethyl]-piperidine;
1-[2-(3,3-dimethyl-2-pyrrolidin-1-ylmethylindan-5-yloxy)-ethyl]-piperidine;
1-[4-(2-pyrrolidin-1-ylmethylindan-5-yloxy)-butyl]-piperidine;
1-[2,2-dimethyl-3-(2-pyrrolidin-1-ylmethylindan-5-yloxy)-propyl]-piperidine;
1-{2-methyl-1-[2-(2-pyrrolidin-1-ylmethylindan-5-yloxy)-ethyl]-propyl)-piperidine;
1-{3-[2-(1-pyrrolidin-1-yl-ethyl)-indan-5-yloxy]-propyl}-piperidine;
4-{1-[5-(3-piperidin-1-ylpropoxy)-indan-2-yl]-ethyl}-thiomorpholine;
4-{1-[5-(3-piperidin-1-ylpropoxy)-indan-2-yl]-ethyl}-thiomorpholine 1-oxide;
4-{1-[5-(3-piperidin-1-ylpropoxy)-indan-2-yl]-ethyl}-thiomorpholine 1,1-dioxide;
N-isopropyl-[5-(3-piperidin-1-ylpropoxy)-indan-2-ylmethyl]-methylamine;
1-[2-(5-methyl-6-pyrrolidin-1-ylmethyl-5,6,7,8-tetrahydro-naphthalen-2-yloxy)-ethyl]-piperidine;
1-[3-(6-methyl-6-pyrrolidin-1-ylmethyl-5,6,7,8-tetrahydro-naphthalen-2-yloxy)-propyl]-piperidine;
N-pyridin-2-yl-[2-(6-pyrrolidin-1-ylmethyl-5,6,7,8-tetrahydro-naphthalen-2-yloxy)-ethyl]-methylamine;
pyrimidin-4-yl-[2-(6-pyrrolidin-1-ylmethyl-5,6,7,8-tetrahydro-naphthalen-2-yloxy)-ethyl]-amine;
1-[3-(6-pyrrolidin-1-ylmethyl-5,6,7,8-tetrahydro-naphthalen-2-yloxy)-propyl]-1,2,3,4-tetrahydro-quinoline;
7-chloro-2-[3-(6-pyrrolidin-1-ylmethyl-5,6,7,8-tetrahydro-naphthalen-2-yloxy)-propyl]-1,2,3,4-tetrahydro-isoquinoline;
2-[3-(3-fluoro-6-pyrrolidin-1-ylmethyl-5,6,7,8-tetrahydro-naphthalen-2-yloxy)-propyl]-5-methoxy-2,3-dihydro-1H-isoindole;
{6-[3-(5,7-dihydropyrrolo[3,4-b]pyridin-6-yl)-propoxy]-1,2,3,4-tetrahydro-naphthalen-2-ylmethyl}-dimethylamine;
6-[2-(6-pyrrolidin-1-ylmethyl-5,6,7,8-tetrahydro-naphthalen-2-yloxy)-ethyl]-5,6,7,8-tetrahydro-[1,6]naphthyridine;
3-[2-(6-pyrrolidin-1-ylmethyl-5,6,7,8-tetrahydro-naphthalen-2-yloxy)-ethyl]-3-azabicyclo[3.3.1]nonane;
7-[2-(6-pyrrolidin-1-ylmethyl-5,6,7,8-tetrahydro-naphthalen-2-yloxy)-ethyl]-7-azabicyclo[2.2.1]heptane; and
1-{6-[2-(2-phenylaziridin-1-yl)-ethoxy]-1,2,3,4-tetrahydro-naphthalen-2-ylmethyl}-pyrrolidine.

11. The compound of formula I, wherein the compound is selected from the group consisting of:
(±)-[3-(6-pyrrolidin-1-ylmethyl-5,6,7,8-tetrahydro-naphthalen-2-yloxy)-propyl]-dimethylamine hydrochloride;
(±)-dimethyl-[2-(6-pyrrolidin-1-ylmethyl-5,6,7,8-tetrahydro-naphthalen-2-yloxy)-ethyl]-amine hydrochloride;
(±)-1-[3-(6-pyrrolidin-1-ylmethyl-5,6,7,8-tetrahydro-naphthalen-2-yloxy)-propyl]-piperidine;
(±)-4-[3-(6-pyrrolidin-1-ylmethyl-5,6,7,8-tetrahydro-naphthalen-2-yloxy)-propyl]-morpholine hydrochloride;
(−)-4-[3-(6-pyrrolidin-1-ylmethyl-5,6,7,8-tetrahydro-naphthalen-2-yloxy)-propyl]-morpholine hydrochloride;
(±)-1-[3-(6-pyrrolidin-1-ylmethyl-5,6,7,8-tetrahydro-naphthalen-2-yloxy)-propyl]-pyrrolidine hydrochloride;
(±)-1′-[3-(6-pyrrolidin-1-ylmethyl-5,6,7,8-tetrahydro-naphthalen-2-yloxy)-propyl]-[1,4′]bi-piperidinyl hydrochloride;
(±)-1-[3-(6-pyrrolidin-1-ylmethyl-5,6,7,8-tetrahydro-naphthalen-2-yloxy)-propyl]-piperidine-4-carboxylic acid amide hydrochloride;
(±)-1-[3-(6-pyrrolidin-1-ylmethyl-5,6,7,8-tetrahydro-naphthalen-2-yloxy)-propyl]-piperidine-3-carboxylic acid amide;
(−)-1-[3-(6-pyrrolidin-1-ylmethyl-5,6,7,8-tetrahydro-naphthalen-2-yloxy)-propyl]-piperidine hydrochloride;
(−)-4-[3-(6-pyrrolidin-1-ylmethyl-5,6,7,8-tetrahydro-naphthalen-2-yloxy)-propyl]-morpholine hydrochloride;
(+)-1-[3-(6-pyrrolidin-1-ylmethyl-5,6,7,8-tetrahydro-naphthalen-2-yloxy)-propyl]-piperidine hydrochloride;
(−)-[3-(6-pyrrolidin-1-ylmethyl-5,6,7,8-tetrahydro-naphthalen-2-yloxy)-propyl]-dimethylamine hydrochloride;
(+)-4-[3-(6-pyrrolidin-1-ylmethyl-5,6,7,8-tetrahydro-naphthalen-2-yloxy)-propyl]-morpholine hydrochloride;
(−)-3,3-dimethyl-1-[3-(6-pyrrolidin-1-ylmethyl-5,6,7,8-tetrahydro-naphthalen-2-yloxy)-propyl]-piperidine hydrochloride;
(−)-[3-(6-pyrrolidin-1-ylmethyl-5,6,7,8-tetrahydro-naphthalen-2-yloxy)-propyl]-spiro[5.5]undec-3-ylamine hydrochloride;
(+)-bicyclo[2.2.1]hept-2-yl-[3-(6-pyrrolidin-1-ylmethyl-5,6,7,8-tetrahydro-naphthalen-2-yloxy)-propyl]-amine hydrochloride;
(+)-3-[3-(6-pyrrolidin-1-ylmethyl-5,6,7,8-tetrahydro-naphthalen-2-yloxy)-propyl]-3-azaspiro-[5.5]undecane hydrochloride;
(−)-cyclopropyl-[3-(6-pyrrolidin-1-ylmethyl-5,6,7,8-tetrahydro-naphthalen-2-yloxy)-propyl]-amine;
(−)-1-{4-phenyl-1-[3-(6-pyrrolidin-1-ylmethyl-5,6,7,8-tetrahydro-naphthalen-2-yloxy)-propyl]-piperidin-4-yl}-ethanone;
(−)-(1,2-dimethylpropyl)-[3-(6-pyrrolidin-1-ylmethyl-5,6,7,8-tetrahydro-naphthalen-2-yloxy)-propyl]-amine;
(+)-1-{3-[3-(6-pyrrolidin-1-yl methyl-5,6,7,8-tetrahydro-naphthalen-2-yloxy)-propylamino]-propyl}-pyrrolidin-2-one;
(−)-1-{3-[3-(6-pyrrolidin-1-ylmethyl-5,6,7,8-tetrahydro-naphthalen-2-yloxy)-propylamino]-propyl}-pyrrolidin-2-one;
(+)-N,N-dimethyl-N′-[3-(6-pyrrolidin-1-ylmethyl-5,6,7,8-tetrahydro-naphthalen-2-yloxy)-propyl]-ethane-1,2-diamine;
(+)-(4-chlorobenzyl)-[3-(6-pyrrolidin-1-ylmethyl-5,6,7,8-tetrahydro-naphthalen-2-yloxy)-propyl]-amine;
(−)-(4-chlorobenzyl)-[3-(6-pyrrolidin-1-ylmethyl-5,6,7,8-tetrahydro-naphthalen-2-yloxy)-propyl]-amine;
(−)-2-ethyl-[3-(6-pyrrolidin-1-ylmethyl-5,6,7,8-tetrahydro-naphthalen-2-yloxy)-propyl]-amino}-ethanol;
(+)-{1-[3-(6-pyrrolidin-1-ylmethyl-5,6,7,8-tetrahydro-naphthalen-2-yloxy)-propyl]-piperidin-3-yl}-methanol;
(+)-(3,4-dichlorobenzyl)-[3-(6-pyrrolidin-1-ylmethyl-5,6,7,8-tetrahydro-naphthalen-2-yloxy)-propyl]-amine;
(−)-(3,4-dichlorobenzyl)-[3-(6-pyrrolidin-1-ylmethyl-5,6,7,8-tetrahydro-naphthalen-2-yloxy)-propyl]-amine;
(+)-benzyl-[3-(6-pyrrolidin-1-ylmethyl-5,6,7,8-tetrahydro-naphthalen-2-yloxy)-propyl]-amine;
(−)-benzyl-[3-(6-pyrrolidin-1-ylmethyl-5,6,7,8-tetrahydro-naphthalen-2-yloxy)-propyl]-amine;
(+)-(2-pyridin-2-ylethyl)-[3-(6-pyrrolidin-1-ylmethyl-5,6,7,8-tetrahydro-naphthalen-2-yloxy)-propyl]-amine;
(−)-(2-pyridin-2-ylethyl)-[3-(6-pyrrolidin-1-ylmethyl-5,6,7,8-tetrahydro-naphthalen-2-yloxy)-propyl]-amine;
(+)-(4-tert-butylcyclohexyl)-[3-(6-pyrrolidin-1-ylmethyl-5,6,7,8-tetrahydro-naphthalen-2-yloxy)-propyl]-amine;
(−)-(4-tert-butylcyclohexyl )-[3-(6-pyrrolidin-1-ylmethyl-5,6,7,8-tetrahydro-naphthalen-2-yloxy)-propyl]-amine;
(+)-(3-methoxybenzyl)-[3-(6-pyrrolidin-1-ylmethyl-5,6,7,8-tetrahydro-naphthalen-2-yloxy)-propyl]-amine;
(−)-(3-methoxybenzyl)-[3-(6-pyrrolidin-1-ylmethyl-5,6,7,8-tetrahydro-naphthalen-2-yloxy)-propyl]-amine;
(+)-(4-fluorobenzyl)-[3-(6-pyrrolidin-1-ylmethyl-5,6,7,8-tetrahydro-naphthalen-2-yloxy)-propyl]-amine;
(−)-(4-fluorobenzyl)-[3-(6-pyrrolidin-1-ylmethyl-5,6,7,8-tetrahydro-naphthalen-2-yloxy)-propyl]-amine;
(+)-(2,6-difluorobenzyl)-[3-(6-pyrrolidin-1-ylmethyl-5,6,7,8-tetrahydro-naphthalen-2-yloxy)-propyl]-amine;
(−)-(2,6-difluorobenzyl)-[3-(6-pyrrolidin-1-ylmethyl-5,6,7,8-tetrahydro-naphthalen-2-yloxy)-propyl]-amine;
(+)-(3-ethoxypropyl)-[3-(6-pyrrolidin-1-ylmethyl-5,6,7,8-tetrahydro-naphthalen-2-yloxy)-propyl]-amine;
(−)-(3-ethoxypropyl)-[3-(6-pyrrolidin-1-ylmethyl-5,6,7,8-tetrahydro-naphthalen-2-yloxy)-propyl]-amine;
(+)-[3-(6-pyrrolidin-1-ylmethyl-5,6,7,8-tetrahydro-naphthalen-2-yloxy)-propyl]-N-cyclohexyl-N-methylamine;
(−)-[3-(6-pyrrolidin-1-ylmethyl-5,6,7,8-tetrahydro-naphthalen-2-yloxy)-propyl]-N-cyclohexyl-N-methylamine;
(+)-(2,2-dimethylpropyl)-[3-(6-pyrrolidin-1-ylmethyl-5,6,7,8-tetrahydro-naphthalen-2-yloxy)-propyl]-amine;
(−)-(2,2-dimethylpropyl)-[3-(6-pyrrolidin-1-ylmethyl-5,6,7,8-tetrahydro-naphthalen-2-yloxy)-propyl]-amine;
(+)-1-cyclohexyl-4-[3-(6-pyrrolidin-1-ylmethyl-5,6,7,8-tetrahydro-naphthalen-2-yloxy)-propyl]-piperazine;
(−)-1-cyclohexyl-4-[3-(6-pyrrolidin-1-ylmethyl-5,6,7,8-tetrahydro-naphthalen-2-yloxy)-propyl]-piperazine;
(+)-1-[3-(6-pyrrolidin-1-ylmethyl-5,6,7,8-tetrahydro-naphthalen-2-yloxy)-propyl]-piperidine-4-carboxylic acid amide;
(−)-1-[3-(6-pyrrolidin-1-ylmethyl-5,6,7,8-tetrahydro-naphthalen-2-yloxy)-propyl]-piperidine-4-carboxylic acid amide;
(+)-1-{4-[3-(6-pyrrolidin-1-ylmethyl-5,6,7,8-tetrahydro-naphthalen-2-yloxy)-propyl]-piperazin-1-yl}-ethanone;
(−)-1-{4-[3-(6-pyrrolidin-1-ylmethyl-5,6,7,8-tetrahydro-naphthalen-2-yloxy)-propyl]-piperazin-1-yl}-ethanone;
(+)-3-methyl-1-[3-(6-pyrrolidin-1-ylmethyl-5,6,7,8-tetrahydro-naphthalen-2-yloxy)-propyl]-piperidine;
(−)-3-methyl-1-[3-(6-pyrrolidin-1-ylmethyl-5,6,7,8-tetrahydro-naphthalen-2-yloxy)-propyl]-piperidine;
(+)-2-methyl-1-[3-(6-pyrrolidin-1-ylmethyl-5,6,7,8-tetrahydro-naphthalen-2-yloxy)-propyl]-piperidine;
(−)-2-methyl-1-[3-(6-pyrrolidin-1-ylmethyl-5,6,7,8-tetrahydro-naphthalen-2-yloxy)-propyl]-piperidine;
(+)-4-methyl-1-[3-(6-pyrrolidin-1-ylmethyl-5,6,7,8-tetrahydro-naphthalen-2-yloxy)-propyl]-piperidine;
(−)-4-methyl-1-[3-(6-pyrrolidin-1-ylmethyl-5,6,7,8-tetrahydro-naphthalen-2-yloxy)-propyl]-piperidine;
(+)-{1-[3-(6-pyrrolidin-1-ylmethyl-5,6,7,8-tetrahydro-naphthalen-2-yloxy)-propyl]-pyrrolidin-3-yl}-diethylamine;
(−)-{1-[3-(p-Pyrrolidin-1-ylmethyl-5,6,7,8-tetrahydro-naphthalen-2-yloxy)-propyl]-pyrrolidin-3-yl}-diethylamine;
(+)-methyl-(1-methylpiperidin-4-yl)-[3-(6-pyrrolidin-1-ylmethyl-5,6,7,8-tetrahydro-naphthalen-2-yloxy)-propyl]-amine;
(−)-methyl-(1-methylpiperidin-4-yl)-[3-(6-pyrrolidin-1-ylmethyl-5,6,7,8-tetrahydro-naphthalen-2-yloxy)-propyl]-amine;
(+)-1-(3,5-dichloropyridin-4-yl)-4-[3-(6-pyrrolidin-1-ylmethyl-5,6,7,8-tetrahydro-naphthalen-2-yloxy)-propyl]-piperazine;
(−)-1-(3,5-dichloropyridin-4-yl)-4-[3-(6-pyrrolidin-1-ylmethyl-5,6,7,8-tetrahydro-naphthalen-2-yloxy)-propyl]-piperazine;
(+)-2-{4-[3-(6-pyrrolidin-1-ylmethyl-5,6,7,8-tetrahydro-naphthalen-2-yloxy)-propyl]-piperazin-1-yl}-pyrimidine;
(−)-2-{4-[3-(6-pyrrolidin-1-ylmethyl-5,6,7,8-tetrahydro-naphthalen-2-yloxy)-propyl]-piperazin-1-yl}-pyrimidine;
(+)-2,2,N,N-tetramethyl-N′-[3-(6-pyrrolidin-1-ylmethyl-5,6,7,8-tetrahydro-naphthalen-2-yloxy)-propyl]-propane-1,3-diamine;
(−)-2,2,N,N-tetramethyl-N′-[3-(6-pyrrolidin-1-ylmethyl-5,6,7,8-tetrahydro-naphthalen-2-yloxy)-propyl]-propane-1,3-diamine;
(+)-2-[3-(6-pyrrolidin-1-ylmethyl-5,6,7,8-tetrahydro-naphthalen-2-yloxy)-propyl]-2,3-dihydro-1H-isoindole;
(−)-2-[3-(6-pyrrolidin-1-ylmethyl-5,6,7,8-tetrahydro-naphthalen-2-yloxy)-propyl]-2,3-dihydro-1H-isoindole;
(+)-{1-[3-(6-pyrrolidin-1-ylmethyl-5,6,7,8-tetrahydro-naphthalen-2-yloxy)-propylamino]-cyclopentyl}-methanol;
(−)-{1-[3-(6-pyrrolidin-1-ylmethyl-5,6,7,8-tetrahydro-naphthalen-2-yloxy)-propylamino]-cyclopentyl}-methanol;
(+)-2,6-dimethyl-4-[3-(6-pyrrolidin-1-ylmethyl-5,6,7,8-tetrahydro-naphthalen-2-yloxy)-propyl]-morpholine;
(−)-2,6-dimethyl-4-[3-(6-pyrrolidin-1-ylmethyl-5,6,7,8-tetrahydro-naphthalen-2-yloxy)-propyl]-morpholine;
(+)-(1-methyl-2-morpholin-4-ylethyl)-[3-(6-pyrrolidin-1-ylmethyl-5,6,7,8-tetrahydro-naphthalen-2-yloxy)-propyl]-amine;
(−)-(1-methyl-2-morpholin-4-ylethyl)-[3-(6-pyrrolidin-1-ylmethyl-5,6,7,8-tetrahydro-naphthalen-2-yloxy)-propyl]-amine;
(+)-cyclohexylmethyl-[3-(6-pyrrolidin-1-ylmethyl-5,6,7,8-tetrahydro-naphthalen-2-yloxy)-propyl]-amine;
(−)-cyclohexylmethyl-[3-(6-pyrrolidin-1-ylmethyl-5,6,7,8-tetrahydro-naphthalen-2-yloxy)-propyl]-amine;
(+)-(4-morpholin-4-yl-tetrahydrofuran-3-yl)-[3-(6-pyrrolidin-1-ylmethyl-5,6,7,8-tetrahydro-naphthalen-2-yloxy)-propyl]-amine;
(−)-(4-morpholin-4-yl-tetrahydrofuran-3-yl)-[3-(6-pyrrolidin-1-ylmethyl-5,6,7,8-tetrahydro-naphthalen-2-yloxy)-propyl]-amine;
(+)-(1-methyl-1-phenylethyl)-[3-(6-pyrrolidin-1-ylmethyl-5,6,7,8-tetrahydro-naphthalen-2-yloxy)-propyl]-amine;
(−)-(1-methyl-1-phenylethyl)-[3-(6-pyrrolidin-1-ylmethyl-5,6,7,8-tetrahydro-naphthalen-2-yloxy)-propyl]-amine;
(+)-furan-2-ylmethyl-[3-(6-pyrrolidin-1-ylmethyl-5,6,7,8-tetrahydro-naphthalen-2-yloxy)-propyl]-methylamine;
(−)-furan-2-ylmethyl-[3-(6-pyrrolidin-1-ylmethyl-5,6,7,8-tetrahydro-naphthalen-2-yloxy)-propyl]-methylamine;
(+)-furan-2-ylmethyl-[3-(6-pyrrolidin-1-ylmethyl-5,6,7,8-tetrahydro-naphthalen-2-yloxy)-propyl]-amine;
(−)-furan-2-ylmethyl-[3-(6-pyrrolidin-1-ylmethyl-5,6,7,8-tetrahydro-naphthalen-2-yloxy)-propyl]-amine;
(+)-2-{1-[3-(6-pyrrolidin-1-ylmethyl-5,6,7,8-tetrahydro-naphthalen-2-yloxy)-propyl]-pyrrolidin-3-yl}-pyridine;
(−)-2-{1-[3-(6-pyrrolidin-1-ylmethyl-5,6,7,8-tetrahydro-naphthalen-2-yloxy)-propyl]-pyrrolidin-3-yl}-pyridine;
(+)-[3-(6-pyrrolidin-1-ylmethyl-5,6,7,8-tetrahydro-naphthalen-2-yloxy)-propyl]-(tetrahydro-pyran-4-ylmethyl)-amine;
(−)-[3-(6-pyrrolidin-1-ylmethyl-5,6,7,8-tetrahydro-naphthalen-2-yloxy)-propyl]-(tetrahydro-pyran-4-ylmethyl)-amine;
(+)-2-{1-[3-(6-pyrrolidin-1-ylmethyl-5,6,7,8-tetrahydro-naphthalen-2-yloxy)-propyl]-pyrrolidin-2-yl}-pyridine;
(−)-2-{1-[3-(6-pyrrolidin-1-ylmethyl-5,6,7,8-tetrahydro-naphthalen-2-yloxy)-propyl]-pyrrolidin-2-yl}-pyridine;
(+)-(1-isopropyl-piperidin-4-ylmethyl)-[3-(6-pyrrolidin-1-ylmethyl-5,6,7,8-tetrahydro-naphthalen-2-yloxy)-propyl]-amine;
(−)-(1-isopropyl-piperidin-4-ylmethyl)-[3-(6-pyrrolidin-1-ylmethyl-5,6,7,8-tetrahydro-naphthalen-2-yloxy)-propyl]-amine;
(+)-[2-(4-fluorophenyl)-ethyl]-[3-(6-pyrrolidin-1-ylmethyl-5,6,7,8-tetrahydro-naphthalen-2-yloxy)-propyl]-amine;
(−)-[2-(4-fluorophenyl)-ethyl]-[3-(6-pyrrolidin-1-ylmethyl-5,6,7,8-tetrahydro-naphthalen-2-yloxy)-propyl]-amine;
(+)-2-methyl-6-[3-(6-pyrrolidin-1-ylmethyl-5,6,7,8-tetrahydro-naphthalen-2-yloxy)-propylamino]-heptan-2-ol;
(−)-2-methyl-6-[3-(6-pyrrolidin-1-ylmethyl-5,6,7,8-tetrahydro-naphthalen-2-yloxy)-propylamino]-heptan-2-ol;
(+)-(1-isopropyl-piperidin-3-ylmethyl)-[3-(6-pyrrolidin-1-ylmethyl-5,6,7,8-tetrahydro-naphthalen-2-yloxy)-propyl]-amine;
(−)-(1-isopropyl-piperidin-3-ylmethyl)-[3-(6-pyrrolidin-1-ylmethyl-5,6,7,8-tetrahydro-naphthalen-2-yloxy)-propyl]-amine;
(+)-[3-(6-pyrrolidin-1-ylmethyl-5,6,7,8-tetrahydro-naphthalen-2-yloxy)-propyl]-cyclohexyl-amine;
(−)-[3-(6-pyrrolidin-1-ylmethyl-5,6,7,8-tetrahydro-naphthalen-2-yloxy)-propyl]-cyclohexyl-amine;
(+)-6-[3-(6-pyrrolidin-1-ylmethyl-5,6,7,8-tetrahydro-naphthalen-2-yloxy)-propyl]-6-azabicyclo-[3.2.1]octane;
(−)-6-[3-(6-pyrrolidin-1-ylmethyl-5,6,7,8-tetrahydro-naphthalen-2-yloxy)-propyl]-6-azabicyclo-[3.2.1]octane;
(+)-4-methoxy-1-[3-(6-pyrrolidin-1-ylmethyl-5,6,7,8-tetrahydro-naphthalen-2-yloxy)-propyl]-piperidine;
(−)-4-methoxy-1-[3-(6-pyrrolidin-1-ylmethyl-5,6,7,8-tetrahydro-naphthalen-2-yloxy)-propyl]-piperidine;
(+)-(2-{1-[3-(6-pyrrolidin-1-ylmethyl-5,6,7,8-tetrahydro-naphthalen-2-yloxy)-propyl]-piperidin-4-yl}-ethyl)-dimethylamine;
(−)-(2-{1-[3-(6-pyrrolidin-1-ylmethyl-5,6,7,8-tetrahydro-naphthalen-2-yloxy)-propyl]-piperidin-4-yl}-ethyl)-dimethylamine;
(+)-2,6-dimethyl-4-{1-[3-(6-pyrrolidin-1-ylmethyl-5,6,7,8-tetrahydro-naphthalen-2-yloxy)-propyl]-piperidin-4-yl}-morpholine;
(−)-2,6-dimethyl-4-{1-[3-(6-pyrrolidin-1-ylmethyl-5,6,7,8-tetrahydro-naphthalen-2-yloxy)-propyl]-piperidin-4-yl}-morpholine;
(+)-2-[3-(6-pyrrolidin-1-ylmethyl-5,6,7,8-tetrahydro-naphthalen-2-yloxy)-propyl]-octahydro-pyrrolo[1,2-a]pyrazine;
(−)-2-[3-(6-pyrrolidin-1-ylmethyl-5,6,7,8-tetrahydro-naphthalen-2-yloxy)-propyl]-octahydro-pyrrolo[1,2-a]pyrazine;
(+)-(1H-imidazol-2-ylmethyl)-methyl-[3-(6-pyrrolidin-1-ylmethyl-5,6,7,8-tetrahydro-naphthalen-2-yloxy)-propyl]-amine;
(−)-(1H-imidazol-2-ylmethyl)-methyl-[3-(6-pyrrolidin-1-ylmethyl-5,6,7,8-tetrahydro-naphthalen-2-yloxy)-propyl]-amine;
(+)-2-(4-fluorophenyl)-1-[3-(6-pyrrolidin-1-ylmethyl-5,6,7-8-tetrahydro-naphthalen-2-yloxy)-propyl]-pyrrolidine;
(−)-2-(4-fluorophenyl)-1-[3-(6-pyrrolidin-1-ylmethyl-5,6,7-8-tetrahydro-naphthalen-2-yloxy)-propyl]-pyrrolidine;
(+)-(2-{1-[3-(6-pyrrolidin-1-ylmethyl-5,6,7,8-tetrahydro-naphthalen-2-yloxy)-propyl]-piperidin-4-yloxy}-ethyl)-dimethylamine;
(−)-(2-{1-[3-(6-pyrrolidin-1-ylmethyl-5,6,7,8-tetrahydro-naphthalen-2-yloxy)-propyl]-piperidin-4-yloxy}-ethyl)-dimethylamine;
(−)-(2-pyrrolidin-1-ylethyl)-[3-(6-pyrrolidin-1-ylmethyl-5,6,7,8-tetrahydro-naphthalen-2-yloxy)-propyl]-amine;
(−)-(2-chlorobenzyl)-[3-(6-pyrrolidin-1-ylmethyl-5,6,7,8-tetrahydro-naphthalen-2-yloxy)-propyl]-amine;
(−)-pyridin-3-ylmethyl-[3-(6-pyrrolidin-1-ylmethyl-5,6,7,8-tetrahydro-naphthalen-2-yloxy)-propyl]-amine;
(−)-(2-piperidin-1-ylethyl)-[3-(6-pyrrolidin-1-ylmethyl-5,6,7,8-tetrahydro-naphthalen-2-yloxy)-propyl]-amine;
(−)-(3,4-difluorobenzyl)-[3-(6-pyrrolidin-1-ylmethyl-5,6,7,8-tetrahydro-naphthalen-2-yloxy)-propyl]-amine;
(−)-N,N,N′-trimethyl-N′-[3-(6-pyrrolidin-1-ylmethyl-5,6,7,8-tetrahydro-naphthalen-2-yloxy)-propyl]-ethane-1,2-diamine;
(−)-(2,4-dichlorobenzyl)-[3-(6-pyrrolidin-1-ylmethyl-5,6,7,8-tetrahydro-naphthalen-2-yloxy)-propyl]-amine;
(−)-(3-imidazol-1-yl-propyl)-[3-(6-pyrrolidin-1-ylmethyl-5,6,7,8-tetrahydro-naphthalen-2-yloxy)-propyl]-amine;
(−)-3,5-dimethyl-1-[3-(6-pyrrolidin-1-ylmethyl-5,6,7,8-tetrahydro-naphthalen-2-yloxy)-propyl]-piperidine;
(−)-1-pyridin-2-yl-4-[3-(6-pyrrolidin-1-ylmethyl-5,6,7,8-tetrahydro-naphthalen-2-yloxy)-propyl]-piperazine;
(−)-(3-azepan-1-ylpropyl)-[3-(6-pyrrolidin-1-ylmethyl-5,6,7,8-tetrahydro-naphthalen-2-yloxy)-propyl]-amine;
(−)-1-[3-(6-pyrrolidin-1-ylmethyl-5,6,7,8-tetrahydro-naphthalen-2-yloxy)-propyl]-1,2,3,4,5,6-hexahydro-[4,4′]bipyridinyl;
(−)-furan-3-ylmethyl-[3-(6-pyrrolidin-1-ylmethyl-5,6,7,8-tetrahydro-naphthalen-2-yloxy)-propyl]-amine;
(−)-1,6-dimethyl-2-[3-(6-pyrrolidin-1-ylmethyl-5,6,7,8-tetrahydro-naphthalen-2-yloxy)-propyl]-1,2,3,4-tetrahydro-pyrrolo[1,2-a]pyrazine;
(−)-[3-(6-pyrrolidin-1-ylmethyl-5,6,7,8-tetrahydro-naphthalen-2-yloxy)-propyl]-[2-(tetrahydro-pyran-2-yl)-ethyl]-amine;
(−)-(2-imidazol-1-yl-ethyl)-[3-(6-pyrrolidin-1-methyl-5,6,7,8-tetrahydro-naphthalen-2-yloxy)-propyl]-amine;
(−)-(1H-imidazol-2-ylmethyl)-[3-(6-pyrrolidin-1-ylmethyl-5,6,7,8-tetrahydro-naphthalen-2-yloxy)-propyl]-amine;
(−)-[3-(6-pyrrolidin-1-ylmethyl-5,6,7,8-tetrahydro-naphthalen-2-yloxy)-propyl]-(tetrahydro-pyran4-ylmethyl)-methylamine; and
(−)-1-[2-(6-pyrrolidin-1-ylmethyl-5,6,7,8-tetrahydro -naphthalen-2-yloxy)-ethyl]-piperidine dihydrochloride.

12. A pharmaceutical composition for treating a disorder or condition that may be treated by antagonizing histamine-3 receptors, the composition comprising a compound of formula I as described in claim 1, and optionally a pharmaceutically acceptable carrier.

13. A method of treatment of a disorder or condition that may be treated by antagonizing histamine-3 receptors, the method comprising administering to a mammal in need of such treatment a compound of formula I as described in claim 1.

14. A pharmaceutical composition comprising a compound of formula I as described in claim 1, and optionally a pharmaceutically acceptable carrier.

15. A method of treatment of a disorder or condition selected from the group consisting of depression, mood disorders, schizophrenia, anxiety disorders, Alzheimer's disease, attention-deficit hyperactivity disorder (ADHD), psychotic disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastro-intestinal tract, the method comprising administering to a mammal in need of such treatment a compound of formula I as described in claim 1.

16. The method of claim 15, wherein the disorder or condition is selected from the group consisting of anxiety disorders, attention-deficit hyperactivity disorder, respiratory diseases, and obesity.

17. The method of claim 15, wherein the disorder or condition is a respiratory disease selected from the group consisting of adult respiratory distress syndrome, acute respiratory distress syndrome, bronchitis, chronic bronchitis, chronic obstructive pulmonary disease, cystic fibrosis, asthma, emphysema, rhinitis and chronic sinusitis.

18. A pharmaceutical composition for treating allergic rhinitis, nasal congestion or allergic congestion comprising:
a) an H3 receptor antagonist compound of formula 1; or a pharmaceutically acceptable salt thereof;
b) an H1 receptor antagonist such as cetirizine; or a pharmaceutically acceptable salt thereof; and
c) a pharmaceutically acceptable carrier;
wherein the active ingredients (a) and (b) above are present in amounts that render the composition effective in treating allergy rhinitis, nasal congestion or allergic congestion

19. A pharmaceutical composition for treating depression and mood disorder comprising:
a) an H3 receptor antagonist or a pharmaceutically acceptable salt thereof;
b) a neurotransmitter uptake blocker or
c) a pharmaceutically acceptable salt thereof;
wherein the active ingredients (a) and (b) above are present in amounts that render the composition effective in treating depression and mood disorder.

20. The composition according to claim 22 wherein the H3 receptor antagonist and the neurotransmitter blocker are given simultaneously.

21. The composition according to claim 21 wherein the H3 receptor antagonist and the H1 receptor antagonist are given simultaneously.

22. The pharmaceutical composition of claim 22 wherein the neurotransmitter uptake blocker are selected from an SSRI; sertraline, fluoxetine and paroxetine.
